(19) United States
(12) Patent Application Publication
(10) Pub. No.: US 2025/0367225 A1
(43) Pub. Date: Dec. 4, 2025

(54) 7DW8-5 TREATMENT FOR COVID-19 AND OTHER VIRUS-INDUCED RESPIRATORY INFECTIONS

(71) Applicant: The Trustees of Columbia University in the City of New York, New York, NY (US)

(72) Inventors: Moriya TSUJI, New York, NY (US); Yaoxing HUANG, Brooklyn, NY (US); Manoj S. NAIR, Fair Lawn, NJ (US); David D. HO, Bedford, NY (US)

(21) Appl. No.: 18/835,512

(22) PCT Filed: Jan. 31, 2023
(86) PCT No.: PCT/US2023/061669

§ 371 Date: Aug. 2, 2024

Related U.S. Application Data
(60) Provisional application No. 63/306,352, filed on Feb. 3, 2022.
(60) Provisional application No. 63/338,292, filed on May 4, 2022.
(60) Provisional application No. 63/354,589, filed on Jun. 22, 2022.

(51) Int. Cl. A61K 31/7032; A61K 9/00; A61P 31/14; A61P 37/04

(52) U.S. Cl. CPC A61K 31/7032; A61K 9/0019; A61K 9/0043; A61P 31/14; A61P 37/04

(57) ABSTRACT

The subject matter described herein relates to methods of zNKT cell activation by the 7DW8-5 glycolipid.

### CROSS-REFERENCE TO RELATED APPLICATIONS

This application claims the benefit of and priority to U.S. Provisional Patent Application No. 63/306,352, filed Feb. 3, 2022, U.S. Provisional Patent Application No. 63/338,292, filed May 4, 2022, and U.S. Provisional Patent Application No. 63/354,589, filed Jun. 22, 2022, the contents of each of which is hereby incorporated by reference in their entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### BACKGROUND OF THE INVENTION

SARS-COV-2, the virus that causes COVID-19, continues to rapidly spread throughout the world's population. Though vaccines have now been developed, rapid mutations of the virus over the past several months have yielded viral variants that current vaccines may not be able to effectively manage. Other prophylactic therapies are lacking.

### SUMMARY OF THE INVENTION

In certain aspects, the invention provides a method of activation of natural killer T (NKT) cells in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue.

In some embodiments, the T cells comprise iNKT cells. In some embodiments, the T cells comprise CD8+ T cells. In some embodiments, the T cells comprise CD4+ T cells.

In some embodiments, the administering is through an intranasal route. In some embodiments, the administering is through an intravenous route. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the activation is mediated via one or more cytokines. In some embodiments, the one or more cytokines is interferon-γ. In some embodiments, the activation of iNKT cells is localized to lungs. In some embodiments, the activation of iNKT cells is localized to nasal turbinates.

In some embodiments, the subject has or is suspected of having a respiratory infection. In some embodiments, the respiratory infection is a SAR-COV-2 infection. In some embodiments, the respiratory infection is a SAR-COV-2 Omicron variant infection. In some embodiments, the respiratory infection is a SAR-COV-2 Delta variant infection. In some embodiments, the respiratory infection is a respiratory syncytial virus (RSV) infection. In some embodiments, the respiratory infection is an influenza infection.

In certain aspects, the invention provides a method of inducing an innate immune response in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue.

In some embodiments, the immune response comprises inducing iNKT cells. In some embodiments, the immune response comprises inducing NK cells. In some embodiments, the immune response comprises inducing CD8+ T cells. In some embodiments, the immune response comprises inducing CD4+ T cells. In some embodiments, the immune response comprises inducing B cells. In some embodiments, the administration is intranasal. In some embodiments, the administration is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the inducing is mediated via one or more cytokines. In some embodiments, the one or more cytokines is interferon-γ. In some embodiments, the inducing is localized to lungs. In some embodiments, the inducing is localized to nasal turbinates.

In some embodiments, the subject has or is suspected of having a respiratory infection. In some embodiments, the respiratory infection is a SAR-COV-2 infection. In some embodiments, the respiratory infection is a SAR-COV-2 Omicron variant infection. In some embodiments, the respiratory infection is a SAR-COV-2 Delta variant infection. In some embodiments, the respiratory infection is a respiratory syncytial virus infection. In some embodiments, the respiratory infection is an influenza infection.

In certain aspects, the invention provides a method of inducing interferon-γ secretion in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue.

In some embodiments, the inducing comprises activation of iNKT cells. In some embodiments, the inducing comprises activation of NK cells. In some embodiments, the inducing comprises activation of CD8+ T cells. In some embodiments, the inducing comprises activation of CD4+ T cells.

In some embodiments, the inducing comprises activation of B cells. In some embodiments, the administering is intranasal. In some embodiments, the administering is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the inducing is localized to lungs. In some embodiments, the inducing is localized to nasal turbinates.

In some embodiments, the subject has or is suspected of having a respiratory infection. In some embodiments, the respiratory infection is a SAR-COV-2 infection. In some embodiments, the respiratory infection is a SAR-COV-2 Omicron variant infection. In some embodiments, the respiratory infection is a SAR-COV-2 Delta variant infection. In some embodiments, the respiratory infection is a respiratory syncytial virus infection. In some embodiments, the respiratory infection is an influenza infection.

In certain aspects, the invention provides a method of treating a respiratory infection in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue. In some embodiments, the method comprises activation of iNKT cells. In some embodiments, the method comprises activation of NK cells. In some embodiments, the method comprises activation of CD8+ T cells. In some embodiments, the method comprises activation of CD4+ T cells. In some embodiments, the method comprises activation of B cells.

In some embodiments, the method comprises increased secretion of one or more cytokines compared to the secretion of the one or more cytokines in a non-treated subject. In some embodiments, the one or more cytokines is interferon-γ.

In some embodiments, the administering is intranasal. In some embodiments, the administering is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the respiratory infection is caused by a SARS-COV-2 virus. In some embodiments, the SARS-COV-2 virus is SARS-COV-2 MA10. In some embodiments, the respiratory infection is cause by an influenza virus. In some embodiments, the influenza virus is PR8.

In some embodiments, the respiratory infection is caused by a SAR-COV-2 Omicron variant virus. In some embodiments, the respiratory infection is caused by a SAR-CoV-2 Delta variant virus. In some embodiments, the respiratory infection is caused by a respiratory syncytial virus.

In certain aspects, the invention provides a method of preventing a respiratory infection in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg. In some embodiments, the glycolipid is α-GalCer. In some embodiments, the glycolipid is an α-GalCer analogue.

In some embodiments, the method comprises activation of iNKT cells. In some embodiments, the method comprises activation of NK cells. In some embodiments, the method comprises activation of CD8+ T cells. In some embodiments, the method comprises activation of CD4+ T cells. In some embodiments, the method comprises activation of B cells.

In some embodiments, the method comprises increased secretion of one or more cytokines compared to the secretion of the one or more cytokines in a non-treated subject. In some embodiments, the one or more cytokines is interferon-γ.

In some embodiments, the administering is intranasal. In some embodiments, the administering is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the respiratory infection is caused by a SARS-COV-2 virus. In some embodiments, the SARS-COV-2 virus is SARS-COV-2 MA10. In some embodiments, the respiratory infection is cause by an influenza virus. In some embodiments, the influenza virus is PR8.

In some embodiments, the respiratory infection is caused by a SAR-COV-2 Omicron variant virus. In some embodiments, the respiratory infection is caused by a SAR-CoV-2 Delta variant virus. In some embodiments, the respiratory infection is caused by a respiratory syncytial virus (RSV).

In certain aspects, the invention provides a method for treating one or more symptoms in a subject having COVID-19, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue.

In some embodiments, the method comprises activation of iNKT cells. In some embodiments, the method comprises activation of NK cells. In some embodiments, the method comprises activation of CD8+ T cells. In some embodiments, the method comprises activation of CD4+ T cells. In some embodiments, the method comprises activation of B cells.

In some embodiments, the method comprises increased secretion of one or more cytokines compared to the secretion of the one or more cytokines in a non-treated subject. In some embodiments, the one or more cytokines is interferon-γ.

In some embodiments, the administering is intranasal. In some embodiments, the administering is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the one or more symptoms are caused by a SARS-COV-2 virus infection. In some embodiments, the SARS-COV-2 virus is a SAR-COV-2 variant virus. In some embodiments, the SARS-COV-2 virus is a SAR-COV-2 Omicron variant virus. In some embodiments, the SARS-COV-2 virus is a SAR-COV-2 Delta variant virus. In some embodiments, the one or more symptoms is fever, chills, cough, shortness of breath, difficulty breathing, fatigue, body aches, headache, loss of taste, loss of smell, sore throat, nasal congestion, nausea, vomiting, or any combination thereof.

### BRIEF DESCRIPTION OF THE FIGURES

The patent or application file contains at least one drawing executed in color. To conform to the requirements for PCT patent applications, many of the figures presented herein are black and white representations of images originally created in color.

FIG. 1 shows the interaction between iNKT cells that carry invTCR, CD1d bearing antigen-presenting cells and glycolipid 7DW8-5.

FIG. 2 shows a schematic representation of the mode of iNKT cell activation by the 7DW8-5 glycolipid and subsequent activation of various immune competent cells.

FIGS. 3A-M show that 7DW8-5 administration blocks infection in mice challenged with 50,000 PFU/mouse of a mouse-adapted SARS-COV-2 viral strain (MA10). FIG. 3A shows body weight change in infected mice treated via various routes. FIG. 3B shows viral titer in lungs of infected mice treated via various routes. FIG. 3C shows body weight change in infected mice administered 7DW8-5 at various time points before infection. FIG. 3D shows viral titer in lungs of infected mice administered 7DW8-5 at various time points before infection. FIG. 3E shows body weight change in infected mice treated with various dosages of 7DW8-5. FIG. 3F shows viral titer in infected mice treated with various dosages of 7DW8-5. FIG. 3G shows viral titter in lungs of infected mice treated with saline or 7DW8-5. FIG. 3H shows viral titter in nasal turbinate of infected mice treated with saline or 7DW8-5. FIG. 3I shows body weight change in infected mice treated saline or 7DW8-5 at multiple time points. FIG. 3J shows viral titer in infected mice treated saline or 7DW8-5 at multiple time points. FIG. 3K shows a schematic representation of the administration schedule in FIGS. 3I-J. FIG. 3L shows that repeat dosing of mice with 7DW8-5 did not reduce its antiviral activity in mice as measured by body weight change. BALB/c mice (N=6) were given 0.2 μg of 7DW8-5 by IN route every 48 h for 10 days before viral challenge. Single dose (0.2 μg) of 7DW8-5 and saline control mice were included (N=6/group). FIG. 3M shows that repeat dosing of mice with 7DW8-5 did not reduce its antiviral activity in mice as measured by lung viral titer.

FIGS. 4A-C show that the prophylactic activity of 7DW8-5 against MA10 is observed in the lungs and the nasal turbinate of mice infected mice. FIG. 4A shows viral titer in lings. FIG. 4B shows viral titer in the nasal turbinate. FIG. 4C shows body weight change in mice.

FIGS. 5A-H show that 7DW8-5 blocks infection by the SARS-COV-2 variants Omicron (B.1.529.1) and Delta (B.1.617.2). FIG. 5A shows weight change in infected mice treated with saline or 7DW8-5. BALB/c mice (N=6) were given 2 μg of 7DW8-5 each intranasally 2 days before intranasal challenge with 150,000 pfu of Omicron strain. Body weight was measured prior to and on day 3 post viral challenge, and the lung viral load was determined 3 days after viral challenge. FIG. 5B shows viral load in infected mice treated with saline or 7DW8-5. FIG. 5C shows PFU/ml in lungs of infected hamsters treated with saline or 7DW8-5. Syrian hamsters (N=5) were given 100 μg/kg of 7DW8-5 each intranasally 2 days before intranasal challenge with 100,000 pfu of Delta strain. FIG. 5D shows PFU/ml in nasal turbine of infected hamsters treated with saline or 7DW8-5. FIGS. 5E-F show the protective effect of 7DW8-5 against SARS-COV-2 Delta variant in K18-transgenic mice. K18 hACE2-Tg C57BL/6 mice (N=10) were given 2 μg of 7DW8-5 each intranasally 2 days before intranasal challenge with 1000 pfu of Delta strain SARS-COV-2. The viral load in the lung and nasal turbinate (NT) was determined 3 days after viral challenge. FIGS. 5G-H show the antiviral activity of 7DW8-5 against influenza virus. C57BL/6 mice (N=7) were given 2 μg of 7DW8-5 each intranasally 2 days before intranasal challenge with 5,000 pfu of influenza A virus PR8 strain. Body weight was measured prior to and on day 3 post viral challenge (FIG. 5G) and the lung viral load determined 3 days after virus challenge (FIG. 5H).

FIGS. 6A-E show that the protective effect of 7DW8-5 is dependent on CD1d-mediated activation. FIG. 6A shows various glycolipids disclosed herein. FIG. 6B shows body weight change in infected mice treated with glycolipids at three days post-infection. FIG. 6C shows viral titer in lungs of infected mice treated with glycolipids at three days post-infection. FIG. 6D shows weight change in infected wild-type or CD1d deficient mice treated with saline or 7DW8-5. FIG. 6E shows viral load in lungs of infected wild-type or CD1d deficient mice treated with saline or 7DW8-5.

FIG. 7 shows lack of body weight loss in 7DW8-5-treated C57BL/6 mice challenged with influenza A virus PR8 strain.

FIGS. 8A-C show that 7DW8-5 is not an anti-SARS-COV-2 compound and that it blocks influenza virus PR8 strain infection. FIG. 8A shows no direct antiviral effect of 7DW8-5 against SARS-COV-2 in VeroE6: Vero E6 cells were treated with indicated doses of drugs in a 96 well plate. After 30 min, SARS-COV-2 (USA/WA1) was added to each well to final MOI 0.1. Cells were incubated at 370C/5% CO2 for 70 h prior to scoring CPE. Percent inhibition was calculated from fold reduction in CPE over control wells receiving no drug. Remdesivir (RDV) was used as a positive control for the assay. Phosphatidylethanolamine (PE) and Biotin-phosphatidylethanolamine (B-PE) were used as a negative control. FIG. 8B shows that 7DW8-5 protects mice from influenza virus. C57BL/6 mice (N=10) were given 2 μg of 7DW8-5 or saline by IN route 2 days before intranasal challenge with 5,000 pfu (lethal dose) of the PR8 strain. Kaplan-Meier Survival curve was plotted. FIG. 8C shows body weight change in mice infected with influenza PR8 virus strain and treated with saline or 7DW8-5.

FIGS. 9A-C show the antiviral activity of 7DW8-5 against RSV-A2. BALB/c mice (N=6) were given 2 μg of 7DW8-5 each intranasally 2 days before intranasal challenge with 600,000 pfu of human RSV A2 strain. Body weight was measured prior to and on day 4 post viral challenge (FIG. 9A) and the lung viral load determined 4 days after the virus challenge (FIG. 9B). FIG. 9C shows body weight changes of individual infected mice treated with saline or 7DW8-5.

FIG. 10 shows that long-term antiviral effect of 7DW8-5 against a MA10 challenge. N=5/group

FIGS. 11A-B show prophylactic activity of 7DW8-5 in K18-hACE2 Tg mice against SARS-COV-2 B.1.617.2 (Delta) variant challenge. FIG. 11A shows plaque-forming unit (PFU) per gram tissue in lungs of infected mice treated with saline or 7DW8-5. FIG. 11B shows plaque-forming unit (PFU) per gram tissue in nasal turbinates of infected mice treated with saline or 7DW8-5. N=10/group

FIGS. 12A-B show relative number of IFN-γ-secreting splenocytes in hamster versus mouse in response to glycolipids in culture. FIG. 12A shows splenocytes in hamster. FIG. 12B shows splenocytes in mouse.

FIGS. 13A-D show differential expression of genes in nasal turbinate of mice following intranasal administration of 7DW8-5. FIG. 13A shows a schematic representation of experimental set-up. FIG. 13B shows RNA expression array 24 hr following intranasal administration of 7DW8-5. FIG. 13C shows a panel of cytokines/chemokines increased in mice upon 7DW8-5 administration. BALB/c mice (N=6) were given 2 μg of 7DW8-5 or saline by IN route, and 24 h later, supernatants were collected by centrifugation of nasal turbinates (NT), bronchoalveolar lavage (BAL) and lung homogenates (LH) of 7DW-8-treated and saline-treated mice. Sera were also collected. The levels of cytokines and chemokines were analyzed by Mouse Cytokine Array/Chemokine Array 31-Plex (MD31) platform (Eve technologies). The amount (pg/mL) of each cytokine/chemokine in the supernatant and sera of mice is shown. The fold increase between the saline and 7DW8-5 treated groups is also shown. FIG. 13D shows Uniform Manifold Approximation and Projection (UMAP) of total 27,532 single CD45+ cells from the lungs of a group of 3 BALB/c mice 24 h after treating with 7DW8-5 (2 μg) or saline by IN (See FIG. 17). The UMAP was split into 15,397 single cells (7DW8-5 treatment, left) and 12,135 single cells (Saline, right). Each cluster (as indicated) was identified by unsupervised clustering and colored. iNKT cell was identified based on the expression of the semi-invariant TCR Vα14-Ja18″.

FIGS. 14A-E shows a list of cytokines/chemokines increased in mice upon 7DW8-5 intranasal administration. FIG. 14A shows a schematic representation of experimental set-up. FIG. 14B shows increase in BAL. FIG. 14C shows increase in nasal turbinate. FIG. 14D shows increase in lung. FIG. 14E shows increase in serum.

FIGS. 15A-E shows that the prophylactic effect of 7DW8-5 against MA10 is primarily mediated by IFN-γ. FIG. 15A shows a schematic representation of 7DW8-5 and antibody administration. FIG. 15B shows body weight changes in infected mice treated with saline, 7DW8-5 and an anti-IFN-γ antibody, 7DW8-5 and a control antibody, and 7DW8-5 alone. FIG. 15C shows viral titer in lings of infected mice treated with saline, 7DW8-5 and an anti-IFN-γ antibody, 7DW8-5 and a control antibody, and 7DW8-5 alone. N=6-7 mice per group. FIGS. 15D-E show that the anti-viral activity of 7DW8-5 in vivo is mediated by IFNγ. C57BL/6 WT or IFNγ KO mice (N-6) were given 2 μg of 7DW8-5 intranasally 2 days before intranasal challenge with 50,000 pfu of SARS-COV-2 MA10 strain. FIG. 15D shows weight change in mice.

FIG. 16 shows induction of a minimal level of IFNγ by 7DW8-5, as determined by ELISA. BALB/c mice (N=5) were given 2 μg of 7DW8-5 intranasally. Twenty-four h later, supernatants were collected by centrifugation of nasal turbinates (NT) and lung homogenates (LH) harvested from 7DW8-5-treated and saline-treated mice. Sera were also collected. The level of IFNγ was then analyzed by Mouse IL-28B/(IFN lambda 3) ELISA (Thermo Fisher). The amount (pg/mL) of each cytokine/chemokine in the supernatant and sera of mice is shown. The fold increase between the saline and 7DW8-5 treated groups is also shown.

FIGS. 17A-B show lung single cell analysis. FIG. 17A shows workflow for the single cell analysis from lung MNCs. FIG. 17B shows violin plots showing the expression levels of representative marker genes across 18 distinct populations.

FIGS. 18A-B show representative 100× images of lungs from 7DW8-5-and saline-treated mice before and after SARS-COV-2 MA10 infection. FIG. 18A shows H&E staining in the top panels. FIG. 18B shows immunohistochemistry (IHC) labeling against SARS-COV-2 nucleocapsid, counterstained with hematoxylin.

FIG. 19 shows an average number of infected spots measured between each treatment group using immunostaining with SARS-COV-2 NP antibody. The number of infected spots were counted in defined image sections of 0.8 mm2 using Image J by setting the threshold at 0 and 145 at 16-bit.

FIG. 20A-B show inhibition of SARS-COV-2 infection of Huh7 cells by supernatant of 7DW8-5-activated human iNKT cells. FIG. 20A shows that human iNKT cell lines from 3 different donors were cocultured with Hela cells transfected with human CD1d in the presence of 7DW8-5 or 18:1 PE or saline for 24 h, and the supernatants were collected and pooled. Amount of IFN-γ in the supernatants was determined by ELISA (inserted Table). Supernatants were then serially diluted (2-fold) and incubated on a monolayer of Huh7 cells overnight. In addition, supernatants were mixed with anti-human IFN-γ antibody (1 μg/mL). Following this incubation, the cells were infected with ic-SARS-COV-2-mNeon isolate and incubated further for 24 h. Cells were examined under fluorescent microscope to enumerate GFP+ cells and percent inhibition was calculated as the difference in fluorescence in the test wells compared to wells receiving no treatment (virus only) and plotted using GraphPad Prism v9.3. FIG. 20B shows that a serial 5-fold dilution of recombinant IFN-γ was used as positive control and added to a monolayer of Huh7 cells and incubated overnight. Subsequently, the cells were infected with ic-SARS-COV-2-mNeon isolate and incubated further for 24 h. Percent inhibition was calculated as described above.

### DETAILED DESCRIPTION OF THE INVENTION

In certain aspects, the invention provides a method of activation of natural killer T (NKT) cells in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue.

In some embodiments, the T cells comprise invariant natural killer T (INKT) cells. In some embodiments, the T cells comprise CD8+ T cells. In some embodiments, the T cells comprise CD4+ T cells.

In some embodiments, the administering is through an intranasal route. In some embodiments, the administering is through an intravenous route. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the activation is mediated via one or more cytokines. In some embodiments, the one or more cytokines is interferon-γ. In some embodiments, the activation of iNKT cells is localized to lungs. In some embodiments, the activation of iNKT cells is localized to nasal turbinates.

In some embodiments, the subject has or is suspected of having a respiratory infection. In some embodiments, the respiratory infection is a SAR-COV-2 infection. In some embodiments, the respiratory infection is a SAR-COV-2 Omicron variant infection. In some embodiments, the respiratory infection is a SAR-COV-2 Delta variant infection. In some embodiments, the respiratory infection is a respiratory syncytial virus (RSV) infection. In some embodiments, the respiratory infection is an influenza infection.

In certain aspects, the invention provides a method of inducing an innate immune response in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue.

In some embodiments, the immune response comprises inducing iNKT cells. In some embodiments, the immune response comprises inducing NK cells. In some embodiments, the immune response comprises inducing CD8+ T cells. In some embodiments, the immune response comprises inducing CD4+ T cells. In some embodiments, the immune response comprises inducing B cells. In some embodiments, the administration is intranasal. In some embodiments, the administration is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the inducing is mediated via one or more cytokines. In some embodiments, the one or more cytokines is interferon-γ. In some embodiments, the inducing is localized to lungs. In some embodiments, the inducing is localized to nasal turbinates.

In some embodiments, the subject has or is suspected of having a respiratory infection. In some embodiments, the respiratory infection is a SAR-COV-2 infection. In some embodiments, the respiratory infection is a SAR-COV-2 Omicron variant infection. In some embodiments, the respiratory infection is a SAR-COV-2 Delta variant infection. In some embodiments, the respiratory infection is a respiratory syncytial virus infection. In some embodiments, the respiratory infection is an influenza infection.

In certain aspects, the invention provides a method of inducing interferon-γ secretion in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue.

In some embodiments, the inducing comprises activation of iNKT cells. In some embodiments, the inducing comprises activation of NK cells. In some embodiments, the inducing comprises activation of CD8+ T cells. In some embodiments, the inducing comprises activation of CD4+ T cells.

In some embodiments, the inducing comprises activation of B cells. In some embodiments, the administering is intranasal. In some embodiments, the administering is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the inducing is localized to lungs. In some embodiments, the inducing is localized to nasal turbinates.

In some embodiments, the subject has or is suspected of having a respiratory infection. In some embodiments, the respiratory infection is a SAR-COV-2 infection. In some embodiments, the respiratory infection is a SAR-COV-2 Omicron variant infection. In some embodiments, the respiratory infection is a SAR-COV-2 Delta variant infection. In some embodiments, the respiratory infection is a respiratory syncytial virus infection. In some embodiments, the respiratory infection is an influenza infection.

In certain aspects, the invention provides a method of treating a respiratory infection in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue. In some embodiments, the method comprises activation of iNKT cells. In some embodiments, the method comprises activation of NK cells. In some embodiments, the method comprises activation of CD8+ T cells. In some embodiments, the method comprises activation of CD4+ T cells. In some embodiments, the method comprises activation of B cells.

In some embodiments, the method comprises increased secretion of one or more cytokines compared to the secretion of the one or more cytokines in a non-treated subject. In some embodiments, the one or more cytokines is interferon-γ.

In some embodiments, the administering is intranasal. In some embodiments, the administering is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the respiratory infection is caused by a SARS-COV-2 virus. In some embodiments, the SARS-COV-2 virus is SARS-COV-2 MA10. In some embodiments, the respiratory infection is cause by an influenza virus. In some embodiments, the influenza virus is PR8.

In some embodiments, the respiratory infection is caused by a SAR-COV-2Omicron variant virus. In some embodiments, the respiratory infection is caused by a SAR-CoV-2 Delta variant virus. In some embodiments, the respiratory infection is caused by a respiratory syncytial virus.

In certain aspects, the invention provides a method of preventing a respiratory infection in a subject in need thereof, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg. In some embodiments, the glycolipid is α-GalCer. In some embodiments, the glycolipid is an α-GalCer analogue.

In some embodiments, the method comprises activation of iNKT cells. In some embodiments, the method comprises activation of NK cells. In some embodiments, the method comprises activation of CD8+ T cells. In some embodiments, the method comprises activation of CD4+ T cells. In some embodiments, the method comprises activation of B cells.

In some embodiments, the method comprises increased secretion of one or more cytokines compared to the secretion of the one or more cytokines in a non-treated subject. In some embodiments, the one or more cytokines is interferon-γ.

In some embodiments, the administering is intranasal. In some embodiments, the administering is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the respiratory infection is caused by a SARS-COV-2 virus. In some embodiments, the SARS-COV-2 virus is SARS-COV-2 MA10. In some embodiments, the respiratory infection is cause by an influenza virus. In some embodiments, the influenza virus is PR8.

In some embodiments, the respiratory infection is caused by a SAR-COV-2 Omicron variant virus. In some embodiments, the respiratory infection is caused by a SAR-CoV-2 Delta variant virus. In some embodiments, the respiratory infection is caused by a respiratory syncytial virus (RSV).

In certain aspects, the invention provides a method for treating one or more symptoms in a subject having COVID-19, the method comprising administering to the subject a pharmaceutical composition comprising a therapeutically effective amount of a glycolipid.

In some embodiments, the glycolipid is 7DW8-5. In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is α-GalCer. In some embodiments, the glycolipid is an α-GalCer analogue.

In some embodiments, the method comprises activation of iNKT cells. In some embodiments, the method comprises activation of NK cells. In some embodiments, the method comprises activation of CD8+ T cells. In some embodiments, the method comprises activation of CD4+ T cells. In some embodiments, the method comprises activation of B cells.

In some embodiments, the method comprises increased secretion of one or more cytokines compared to the secretion of the one or more cytokines in a non-treated subject. In some embodiments, the one or more cytokines is interferon-γ.

In some embodiments, the administering is intranasal. In some embodiments, the administering is intravenous. In some embodiments, the administering is through an oral route. In some embodiments, the administering is through an intramuscular route. In some embodiments, the administering is through a subcutaneous route. In some embodiments, the administering is through an intradermal route. In some embodiments, the intradermal route comprises one of more microneedle patches.

In some embodiments, the one or more symptoms are caused by a SARS-COV-2 virus infection. In some embodiments, the SARS-COV-2 virus is a SAR-COV-2 variant virus. In some embodiments, the SARS-COV-2 virus is a SAR-COV-2 Omicron variant virus. In some embodiments, the SARS-COV-2 virus is a SAR-COV-2 Delta variant virus. In some embodiments, the one or more symptoms is fever, chills, cough, shortness of breath, difficulty breathing, fatigue, body aches, headache, loss of taste, loss of smell, sore throat, nasal congestion, nausea, vomiting, or any combination thereof.

In some embodiments, the subject matter described herein relates to a method of activating the immune system in a subject in need thereof, the method comprising administering to the subject a therapeutically effective amount of 7DW8-5. In some embodiments, the subject matter described herein relates to a method of activating innate T cells, namely natural killer T (NKT) cells, in a subject in need thereof, the method comprising administering to the subject a therapeutically effective amount of 7DW8-5. In some embodiments, the subject matter described herein relates to a method of mitigating respiratory virus infection in a subject in need thereof, the method comprising administering to the subject a therapeutically effective amount of 7DW8-5. In some embodiments, the subject matter described herein relates to a method of treating respiratory infection. In some embodiments, the respiratory infection is an upper respiratory infection. In some embodiments, the respiratory infection is caused by a virus. In some embodiments, the virus is a mouse-adapted SARS-COV-2 strain, MA10. In some embodiments, the virus is SARS-CoV-2 Omicron variant. In some embodiments the virus is an influenza virus. In some embodiments the virus is a H1N1 influenza virus. In some embodiments, the influenza virus is a mouse-adapted H1N1 influenza virus strain, PR8. In some embodiments, the virus is respiratory syncytial virus (RSV). In some embodiments, the subject matter described herein relates to a method of activating iNKT cells in a subject in need thereof, the method comprising administering to the subject a therapeutically effective amount of 7DW8-5. In some embodiments, the subject matter described herein relates to a method of inducing cytokine secretion in a subject in need thereof, the method comprising administering to the subject a therapeutically effective amount of 7DW8-5. In some embodiments, the cytokine is interferon-γ (IFN-γ). In some embodiments, IFN-γ is secreted by activated iNKT cells. In some embodiments, the activation of iNKT cells is mediated by CD1d.

### The Immune Response

The innate immune response, as opposed to adaptive immunity, is a pathogen non-specific response of the immune system, which recruits immune cells to infection sites in the body by producing chemical factors, including cytokines. Activation of T cells can play a crucial role in the body's immune response against viral infections (1). In some embodiments, the activation of invariant natural killer T (iNKT) cells leads to the subsequent activation of other immune competent cells, including but not limited to, natural killer (NK) cells and CD8+ T cells (2,3). Upon activation, these cells can secrete cytokines. For example, activated CD8+ T cells can induce cell death of virus-infected cells via the production of cytokines, such as interferon-γ (1). Additionally, interferon-γ can induce an antiviral state in uninfected cells (1). Patients with COVID-19 or who have had COVID-19 exhibit higher activation of T-cells (4).

Invariant Natural Killer T (iNKT) Cells

Invariant natural killer T (iNKT) cells are a sub-population of T cells that express an invariant αβT-cell receptor (TCR) and a number of cell surface molecules in common with natural killer (NK) cells. These cells are also known as type I or classical NKT cells. iNKT cells are rare in the human blood pool, comprising only 0.01-1% of peripheral blood mononuclear cells (PBMCs). However, it is noteworthy that unlike conventional T cells that express up to 10¹⁵ different TCRs, iNKT cells express only one invariant TCR (Vα24-JαQ in humans and Vα14-Jα281 in mice), and, therefore, the precursor frequency of iNKT cells ends up extremely high compared to that of conventional T cells. The unique property of iNKT cells makes them important immunoregulatory cells capable of rapidly producing large amounts of various cytokines that can influence other immune cells. iNKT cells can recognize glycolipid antigens presented by the non-polymorphic MHC class I-like molecule, CD1d. iNKT cells recognize the prototypical glycolipid, α-galactosylceramide (α-GalCer), which is derived from bacteria living in marine-sponge.

Human iNKT cells can be divided functionally into three subpopulations: CD4+, CD8+ or CD4-CD8-double negative (DN) cells. CD4+ iNKT cells produce both Th1 and Th2-type cytokines and may have a more immunoregulatory role, while CD8+ and DN iNKT cells are more Th1-like in response and have a stronger cytolytic ability. iNKT cell-derived cytokines and chemokines can modulate several other cell types, including natural killer cells, macrophages, neutrophils, and B cells as well as recruiting and activating dendritic cells.

### CD1 Molecules

CD1 molecules are a family of conserved antigen presenting proteins. These proteins are similar in function to classical MHC molecules, however, while classical MHC molecules present peptides, CD1 proteins bind a variety of lipids and glycolipids and present them to T lymphocytes. There are five known isoforms of CD1 molecules classified into two groups, group I (CD1a, CD1b, CD1c, and CD1e in humans) and group II (CD1d in humans and mice). These groupings are based on similarities in nucleotide and amino acid sequences.

The crystal structure of murine (m) CD1d shows that it adopts a folded conformation related to major histocompatibility complex (MHC) class I proteins. Additionally, mCD1d is suggested to accommodate long lipid tails in two hydrophobic pockets, designated A′ and F′, located in the binding groove. It been demonstrated that CD1d, when loaded with antigenic glycolipids, binds the lipid portion in a hydrophobic groove while making available the hydrophilic sugar moiety to make contact with the T-cell receptor. (6) Λ-Galactosylceramide (α-GalCer), a lipid found in bacteria, Sphingomonas alaskensis, which live in the marine sponge Agellas mauritianus, is the most extensively studied ligand for CD1d. Λ-GalCer, when bound to CD1d, stimulates rapid Th1 and Th2 cytokine production by Vα14i natural killer T (Vα14i NKT) cells in mice and the human homologue Vα24i NKT cells.

In some embodiments, the subject matter described herein relates to the administration of a glycolipid which activates the “innate” immune system. In some embodiments, the administration is intranasal. In some embodiments, the glycolipid is α-GalCer or an α-GalCer analog. In some embodiments, α-GalCer binds to CD1d in order to activate natural killer (NKT) T cells. In some embodiments, the NKT cells are invariant natural killer T (iNKT) cells. In some embodiments, activation of NKT cells leads to cytokine secretion. In some embodiments, activation of NKT cells leads to IFN-γ secretion. In some embodiments, IFN-γ secretion leads to DCs maturation.

In some embodiments, α-GalCer or the α-GalCer analogue provides protection against SARS-COV-2. In some embodiments, α-GalCer or the α-GalCer analogue provides protection against SARS-COV-2 Omicron variant infection. In some embodiments, α-GalCer or the α-GalCer analogue provides protection against SARS-COV-2 Delta variant infection. In some embodiments, α-GalCer or the α-GalCer analogue provides a broad protection against all SARS-COV-2 variants. In some embodiments, α-GalCer or the α-GalCer analogue provides protection against all respiratory viral infections. In some embodiments, α-GalCer or the α-GalCer analogue provides protection against influenza virus infections. In some embodiments, α-GalCer or the α-GalCer analogue provides protection against respiratory syncytial virus (RSV).

In some embodiments, α-GalCer or an α-GalCer analogue is administered to treat a viral respiratory infection in a subject in need thereof. In some embodiments, α-GalCer or an α-GalCer analogue is administered to prevent a viral respiratory infection in a subject in need thereof. In some embodiments, the viral respiratory infection is caused by a coronavirus. In some embodiments, the viral respiratory infection is caused by a SARS-COV-2 virus. In some embodiments, the viral respiratory infection is caused by a SARS-COV-2 variant virus. In some embodiments, the viral respiratory infection is caused by a SARS-COV-2 Omicron variant virus. In some embodiments, the viral respiratory infection is caused by a SARS-CoV-2 Delta variant virus. In some embodiments, the viral respiratory infection is caused by an RSV virus. In some embodiments, the viral respiratory infection is caused by an influenza virus.

### 7DW8-5

7DW8-5 is a glycolipid consisting of two lipid tails and a galactose head, which are o-linked. The chemical formula of 7DW8-5 is C₄₁H₇₂FNO₉. Its molecular weight is 742.01 kDa. The systematic name of 7DW8-5 is (2S, 3S, 4R)-1-O-(α-D-galactopyranosyl)-N-(11-(4-fluorophenyl) undecanoyl)-2-amino-1,3,4-octadecanetriol). 7DW8-5 is modified from α-GalCer.

7DW8-5 binds an MHC class-I like molecule, CD1d, and activates innate T cells, which are iNKT cells, through their invariant T-cell receptor (5). Upon activation, iNKT cells can secrete a large number of cytokines that include interferon-γ (IFN-γ), which is known to have anti-viral activity (5). Activation of iNKT cells rapidly induces activation and maturation of dendritic cells (DCs), which in turn induce a cascade of various immune competence cells, such as natural killer (NK) cells and CD8+ T cells (2, 3). These NK cells and CD8+ T cells can also produce a large amount of IFN-γ.

In some embodiments, the subject matter described herein relates to the administration of a glycolipid which activates the “innate” immune system rather than a pathogen-specific immune response. In some embodiments, the administration is intranasal. In some embodiments, the glycolipid is 7DW8-5. In some embodiments, 7DW8-5 binds to CD1d in order to activate innate invariant natural killer T (iNKT) cells. In some embodiments, activation of iNKT cells leads to maturation of DCs. In some embodiments, activation of iNKT cells leads to cytokine secretion. In some embodiments, activation of iNKT cells leads to IFN-γ secretion. In some embodiments, IFN-γ secretion leads to DCs maturation. In some embodiments, 7DW8-5 administration induces the activation of natural killer cells and CD8+ T cells.

In some embodiments, 7DW8-5 provides protection against SARS-COV-2. In some embodiments, 7DW8-5 provides protection against SARS-COV-2 Omicron variant infection. In some embodiments, 7DW8-5 provides protection against SARS-COV-2 Delta variant infection. In some embodiments, 7DW8-5 can provide a broad protection against all SARS-COV-2 variants. In some embodiments, 7DW8-5 provides protection against all respiratory viral infections. In some embodiments, 7DW8-5 provides protection against influenza virus infections. In some embodiments, 7DW8-5 administration provides protection against RSV.

In some embodiments, 7DW8-5 is administered to treat a viral respiratory infection in a subject in need thereof. In some embodiments, 7DW8-5 is administered to prevent a viral respiratory infection in a subject in need thereof. In some embodiments, the viral respiratory infection caused by a coronavirus. In some embodiments, the viral respiratory infection is caused by a SARS-COV-2 virus. In some embodiments, the viral respiratory infection is caused by a SARS-COV-2 variant virus. In some embodiments, the viral respiratory infection is caused by a SARS-COV-2 Omicron variant virus. In some embodiments, the viral respiratory infection is caused by a SARS-COV-2 Delta variant virus. In some embodiments, the viral respiratory infection is caused by an RSV virus. In some embodiments, the viral respiratory infection is caused by an influenza virus.

In some embodiments, the subject matter described herein relates to a method of activation of natural killer T (NKT) cells in a subject in need thereof, the method comprising administering to the subject a pharmaceutical amount of a glycolipid. In some embodiments, the glycolipid is 7DW8-5. In some embodiments, the glycolipid is alpha-GalCer. In some embodiments, the glycolipid is an alpha-GalCer or a 7DW8-5. In some embodiments, the analog has stimulatory activity against iNKT cells. In some embodiments, the analogue is a 4″-modified α-galactosylceramide. In some embodiments, the analogue is a 6″-triazole-substituted α-galactosyl ceramide. In some embodiments, the analogue is an acyl-chain-and galactose-6″-modified analogue of α-GalCer. In some embodiments, the analogue is a c-glycoside analogue of α-galactosylceramide. In some embodiments, the analogue is an α-galactosylceramide with amide-linked phenyl alkane substitutions on the C4″ position of the galactose ring. In some embodiments, the analogue is a C-5″ and C-6″-modified α-GalCer. In some embodiments, the analogue is KRN7000 (alpha-GalCer). In some embodiments, the analogue is AH10-7. In some embodiments, the analogue is AH15-1. In some embodiments, the analogue is an α-galactosylsphingamides. In some embodiments, the analogue is a n-acyl variant of α-galactosylceramides. In some embodiments, the analogue is a non-glycosidic analogue. In some embodiments, the analogue is ThrCer 6.

Additional analogs and compounds, which can be used with the methods described here can be found in Janssens, J. et al., ACS Med. Chem. Lett. 2017, 8, 642-647, Jervis, P. J. et al., Bioorg. Med. Chem. Lett. 2012, 22, 13, 4348-4352, Hsieh, M. H. et al., Chembiochem. 2012, 13, 11, 1689-1697, Schmieg, J. et al., J. Exp. Med. 2003, 198, 11, 1631-1641, Tyznik, A. J. et al., Chem Biol. 2011, 18, 12, 1620-1630, Saavedra-Avila, N. A. et al., ACS Chem Biol. 2020, 15, 12, 3176-3186, Kharkwal, S. S. et al., Cancer Res. 2021, 81, 7, 1788-1801, Guillaume, J. et al., Bioorg Med Chem. 2015, 23, 13, 3175-3182,Chennamadhavuni, D. et al., Cell Chemical Biology 2018, 25, 571-584, Guillaume, J. et al., Scientific Reports 2017, 7, 4276, 1-18, Yu, K. O. A. et al., PNAS 2005, 102, 9, 3383-3388, Jukes, J. P. et al., Eur. J. Immunol. 2016. 46, 1224-1234, Veerapen, N. et al., Bioconjug Chem. 2018, 29, 9, 3161-3173, and Hartrampf, N. et al., Chemistry. 2020, 26, 20, 4476-4479, the contents of each of which is incorporated herein by reference.

### Administration of the Glycolipid

In some embodiments, the glycolipid described herein is administered intranasally. Nasal drug administration is an effective route of administration because there is a large surface area for absorption of the drug, porous endothelial membrane, high total blood flow, and the routes avoids first-pass metabolism. In some embodiments, any formulation, method, or device of intranasal administration known in the art can be used to administer the glycolipid.

In some embodiments, the intranasal administration of the glycolipid is via an aerosol. In some embodiments, an aerosol is a suspension of the glycolipid in the air. In some embodiments, an aerosol refers to the particlization or atomization of a formulation of the glycolipid and its suspension in the air. In some embodiments, an aerosol formulation is a formulation comprising the glycolipid for nasal inhalation or pulmonary administration.

In some embodiments, the intranasal administration of the glycolipid is via an inhaler. In some embodiments, an inhaler is a device for nasal and/or pulmonary administration of a drug. In some embodiments, the drug in the inhaler is in a solution formulation, a powder formulation, or an aerosol formulation. In some embodiments, the inhaler comprises a propellant-driven inhaler, such as is used for to administer antihistamine for acute asthma attacks. In some embodiments, the inhaler comprises a plastic spray bottle, such as are used to administer decongestants.

In some embodiments, the intranasal administration of the glycolipid is via a dispersant. In some embodiments, the dispersant comprises an agent that assists aerosolization of the glycolipid or absorption of the glycolipid in mucosal tissue, or both. In some embodiments, the dispersant can be a mucosal penetration enhancer. In some embodiments, the dispersant is pharmaceutically acceptable. In some embodiments, “pharmaceutically acceptable” refers to approved by a regulatory agency of the Federal or a state government or listed in the U.S. Pharmacopeia or other generally recognized pharmacopeia for use in animals and in humans. Suitable dispersing agents are well known in the art, and include but are not limited to surfactants. Surfactants can reduce surface induced aggregation of drugs caused by atomization of the solution forming the liquid aerosol. Examples of surfactants include, but are not limited to, polyoxyethylene fatty acid esters and alcohols, and polyoxyethylene sorbitan fatty acid esters. Amounts of surfactants used will vary, being generally within the range or 0.001 and 4% by weight of the formulation. Surfactants can also be selected on the basis of desired properties, depending on the specific formulation, concentration of the glycolipid, diluent (in a liquid formulation) or form of powder (in a dry powder formulation).

In some embodiments, the glycolipid is formulated as oral or parenteral dosage forms, which are administered orally, such as uncoated tablets, coated tablets, pills, capsules, powders, granulates, dispersions or suspensions. In some embodiments, the glycolipid is administered intravenously. In some embodiments, the glycolipid is formulated for intravenous administration, for example, an IV bag. In some embodiments, the glycolipid is administered topically. In some embodiments, the glycolipid is formulated in ointment, cream or gel form for transdermal administration. In some embodiments, the glycolipid is formulated as an aerosol or spray for nasal application. In another embodiment, the glycolipid is formulated in a liquid dosage form. Examples of suitable liquid dosage forms include solutions or suspensions in water, pharmaceutically acceptable fats and oils, alcohols or other organic solvents, including esters, emulsions, syrups or elixirs, solutions and/or suspensions. In some embodiments, suitable excipients and carriers are solid or liquid and the type is generally chosen based on the type of administration being used. Liposomes may also be used to deliver the composition. Examples of suitable solid carriers include lactose, sucrose, gelatin and agar. Oral dosage forms may contain suitable binders, lubricants, diluents, disintegrating agents, coloring agents, flavoring agents, flow-inducing agents, and melting agents. Liquid dosage forms may contain suitable solvents, preservatives, emulsifying agents, suspending agents, diluents, sweeteners, thickeners, and melting agents. Parenteral and intravenous forms can also include minerals and other materials to make them compatible with the type of injection or delivery system chosen.

In some embodiments, the glycolipid is administered through an oral route. In some embodiments, the glycolipid is administered through an intramuscular route. In some embodiments, the glycolipid is administered through a subcutaneous route. In some embodiments, the glycolipid is administered through an intradermal route. In some embodiments, the intradermal route comprises one of more microneedle patches. In some embodiments, the glycolipid is administered through an intravenous route.

In some embodiments, the glycolipid is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the glycolipid is administered in a single dosage or a cumulative dosage, such as a serial dosage. In some embodiments, the dosage and regimen of the administration of the glycolipid can be readily determined by one skilled in the art. Treatment of viral infections may include multiple administrations of a pharmaceutical composition comprising a therapeutically effective amount of the glycolipid. The administration can be carried out over a range of time periods, such as once daily, twice daily, trice daily, once every few days, or once weekly. The timing of administration can vary among subjects, depending on the severity of an symptoms and/or viral load. In some embodiments, an effective dose of the pharmaceutical composition comprising the glycolipid is once or multiple times daily until the subject no longer requires therapy or disease symptoms have resolved. In some embodiments, the subject can be monitored throughout the course of treatment and that the dosage of the pharmaceutical composition disclosed herein that is administered can be adjusted according to treatment progress.

In some embodiments, the glycolipid described herein is α-GalCer or an α-GalCer analogue. In some embodiments, the α-GalCer or α-GalCer analogue is administered intranasally. Nasal drug administration is an effective route of administration because there is a large surface area for absorption of the drug, porous endothelial membrane, high total blood flow, and the routes avoids first-pass metabolism. In some embodiments, any formulation, method, or device of intranasal administration known in the art can be used to administer the α-GalCer or α-GalCer analogue.

In some embodiments, the intranasal administration of the α-GalCer or α-GalCer analogue is via an aerosol. In some embodiments, an aerosol is a suspension of the α-GalCer or α-GalCer analogue in the air. In some embodiments, an aerosol refers to the particlization or atomization of a formulation of the α-GalCer or α-GalCer analogue and its suspension in the air. In some embodiments, an aerosol formulation is a formulation comprising the α-GalCer or α-GalCer analogue for nasal inhalation or pulmonary administration.

In some embodiments, the intranasal administration of the α-GalCer or α-GalCer analogue is via an inhaler. In some embodiments, an inhaler is a device for nasal and/or pulmonary administration of a drug. In some embodiments, the drug in the inhaler is in a solution formulation, a powder formulation, or an aerosol formulation. In some embodiments, the inhaler comprises a propellant-driven inhaler, such as is used for to administer antihistamine for acute asthma attacks. In some embodiments, the inhaler comprises a plastic spray bottle, such as are used to administer decongestants.

In some embodiments, the intranasal administration of the α-GalCer or α-GalCer analogue is via a dispersant. In some embodiments, the dispersant comprises an agent that assists aerosolization of the α-GalCer or α-GalCer analogue or absorption of the α-GalCer or α-GalCer analogue in mucosal tissue, or both. In some embodiments, the dispersant can be a mucosal penetration enhancer. In some embodiments, the dispersant is pharmaceutically acceptable. In some embodiments, “pharmaceutically acceptable” refers to approved by a regulatory agency of the Federal or a state government or listed in the U.S. Pharmacopeia or other generally recognized pharmacopeia for use in animals and in humans. Suitable dispersing agents are well known in the art, and include but are not limited to surfactants. Surfactants can reduce surface induced aggregation of drugs caused by atomization of the solution forming the liquid aerosol. Examples of surfactants include, but are not limited to, polyoxyethylene fatty acid esters and alcohols, and polyoxyethylene sorbitan fatty acid esters. Amounts of surfactants used will vary, being generally within the range or 0.001 and 4% by weight of the formulation. Surfactants can also be selected on the basis of desired properties, depending on the specific formulation, concentration of the α-GalCer or α-GalCer analogue, diluent (in a liquid formulation) or form of powder (in a dry powder formulation).

In some embodiments, the α-GalCer or α-GalCer analogue is formulated as oral or parenteral dosage forms, which are administered orally, such as uncoated tablets, coated tablets, pills, capsules, powders, granulates, dispersions or suspensions. In some embodiments, the α-GalCer or α-GalCer analogue is administered intravenously. In some embodiments, the α-GalCer or α-GalCer analogue is formulated for intravenous administration, for example, an IV bag. In some embodiments, the α-GalCer or α-GalCer analogue is administered topically. In some embodiments, the α-GalCer or α-GalCer analogue is formulated in ointment, cream or gel form for transdermal administration. In some embodiments, the α-GalCer or α-GalCer analogue is formulated as an aerosol or spray for nasal application. In another embodiment, the α-GalCer or α-GalCer analogue is formulated in a liquid dosage form. Examples of suitable liquid dosage forms include solutions or suspensions in water, pharmaceutically acceptable fats and oils, alcohols or other organic solvents, including esters, emulsions, syrups or elixirs, solutions and/or suspensions. In some embodiments, suitable excipients and carriers are solid or liquid and the type is generally chosen based on the type of administration being used. Liposomes may also be used to deliver the composition. Examples of suitable solid carriers include lactose, sucrose, gelatin and agar. Oral dosage forms may contain suitable binders, lubricants, diluents, disintegrating agents, coloring agents, flavoring agents, flow-inducing agents, and melting agents. Liquid dosage forms may contain suitable solvents, preservatives, emulsifying agents, suspending agents, diluents, sweeteners, thickeners, and melting agents. Parenteral and intravenous forms can also include minerals and other materials to make them compatible with the type of injection or delivery system chosen.

In some embodiments, the α-GalCer or α-GalCer analogue is administered through an oral route. In some embodiments, the α-GalCer or α-GalCer analogue is administered through an intramuscular route. In some embodiments, the α-GalCer or α-GalCer analogue is administered through a subcutaneous route. In some embodiments, the α-GalCer or α-GalCer analogue is administered through an intradermal route. In some embodiments, the intradermal route comprises one of more microneedle patches. In some embodiments, the α-GalCer or α-GalCer analogue is administered through an intravenous route.

In some embodiments, the α-GalCer or α-GalCer analogue is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the α-GalCer or α-GalCer analogue is administered in a single dosage or a cumulative dosage, such as a serial dosage. In some embodiments, the dosage and regimen of the administration of the α-GalCer or α-GalCer analogue can be readily determined by one skilled in the art. Treatment of viral infections may include multiple administrations of a pharmaceutical composition comprising a therapeutically effective amount of the α-GalCer or α-GalCer analogue. The administration can be carried out over a range of time periods, such as once daily, twice daily, trice daily, once every few days, or once weekly. The timing of administration can vary among subjects, depending on the severity of an symptoms and/or viral load. In some embodiments, an effective dose of the pharmaceutical composition comprising the α-GalCer or α-GalCer analogue is once or multiple times daily until the subject no longer requires therapy or disease symptoms have resolved. In some embodiments, the subject can be monitored throughout the course of treatment and that the dosage of the pharmaceutical composition disclosed herein that is administered can be adjusted according to treatment progress.

In some embodiments, the glycolipid described herein is 7DW8-5. In some embodiments, 7DW8-5 is administered intranasally. Nasal drug administration is an effective route of administration because there is a large surface area for absorption of the drug, porous endothelial membrane, high total blood flow, and the routes avoids first-pass metabolism. In some embodiments, any formulation, method, or device of intranasal administration known in the art can be used to administer 7DW8-5.

In some embodiments, the intranasal administration of 7DW8-5 is via an aerosol. In some embodiments, an aerosol is a suspension of 7DW8-5 in the air. In some embodiments, aerosol refers to the particlization or atomization of a formulation of 7DW8-5 and its suspension in the air. In some embodiments, an aerosol formulation is a formulation comprising 7DW8-5 for nasal inhalation or pulmonary administration.

In some embodiments, the intranasal administration of 7DW8-5 is via an inhaler. In some embodiments, an inhaler is a device for nasal and/or pulmonary administration of a drug. In some embodiments, the drug in the inhaler is in a solution formulation, a powder formulation, or an aerosol formulation. In some embodiments, the inhaler comprises a propellant-driven inhaler, such as is used for to administer antihistamine for acute asthma attacks. In some embodiments, the inhaler comprises a plastic spray bottle, such as are used to administer decongestants.

In some embodiments, the intranasal administration of 7DW8-5 is via a dispersant. In some embodiments, the dispersant comprises an agent that assists aerosolization of 7DW8-5 or absorption of 7DW8-5 in mucosal tissue, or both. In some embodiments, the dispersant can be a mucosal penetration enhancer. In some embodiments, the dispersant is pharmaceutically acceptable. In some embodiments, “pharmaceutically acceptable” refers to approved by a regulatory agency of the Federal or a state government or listed in the U.S. Pharmacopeia or other generally recognized pharmacopeia for use in animals and in humans. Suitable dispersing agents are well known in the art, and include but are not limited to surfactants. Surfactants can reduce surface induced aggregation of drugs caused by atomization of the solution forming the liquid aerosol. Examples of surfactants include, but are not limited to, polyoxyethylene fatty acid esters and alcohols, and polyoxyethylene sorbitan fatty acid esters. Amounts of surfactants used will vary, being generally within the range or 0.001 and 4% by weight of the formulation. Surfactants can also be selected on the basis of desired properties, depending on the specific formulation, concentration of 7DW8-5, diluent (in a liquid formulation) or form of powder (in a dry powder formulation).

In some embodiments, 7DW8-5 is formulated as oral or parenteral dosage forms, which are administered orally, such as uncoated tablets, coated tablets, pills, capsules, powders, granulates, dispersions or suspensions. In some embodiment, 7DW8-5 is administered intravenously. In some embodiments, 7DW8-5 is formulated for intravenous administration, for example, an IV bag. In some embodiments, 7DW8-5 is administered typically. In some embodiments, 7DW8-5 is formulated in ointment, cream or gel form for transdermal administration. In some embodiments, 7DW8-5 is formulated as an aerosol or spray for nasal application. In another embodiment, 7DW8-5 is formulated in a liquid dosage form. Examples of suitable liquid dosage forms include solutions or suspensions in water, pharmaceutically acceptable fats and oils, alcohols or other organic solvents, including esters, emulsions, syrups or elixirs, solutions and/or suspensions. In some embodiments, suitable excipients and carriers are solid or liquid and the type is generally chosen based on the type of administration being used. Liposomes may also be used to deliver the composition. Examples of suitable solid carriers include lactose, sucrose, gelatin and agar. Oral dosage forms may contain suitable binders, lubricants, diluents, disintegrating agents, coloring agents, flavoring agents, flow-inducing agents, and melting agents. Liquid dosage forms may contain suitable solvents, preservatives, emulsifying agents, suspending agents, diluents, sweeteners, thickeners, and melting agents. Parenteral and intravenous forms can also include minerals and other materials to make them compatible with the type of injection or delivery system chosen.

In some embodiments, 7DW8-5 is administered through an oral route. In some embodiments, 7DW8-5 is administered through an intramuscular route. In some embodiments, 7DW8-5 is administered through a subcutaneous route. In some embodiments, 7DW8-5 is administered through an intradermal route. In some embodiments, the intradermal route comprises one of more microneedle patches. In some embodiments, 7DW8-5 is administered through an intravenous route.

In some embodiments, 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

In some embodiments, the 7DW8-5 is administered in a single dosage or a cumulative dosage, such as a serial dosage. In some embodiments, the dosage and regimen of the administration of the 7DW8-5 can be readily determined by one skilled in the art. Treatment of viral infections may include multiple administrations of a pharmaceutical composition comprising a therapeutically effective amount of the 7DW8-5. The administration can be carried out over a range of time periods, such as once daily, twice daily, trice daily, once every few days, or once weekly. The timing of administration can vary among subjects, depending on the severity of an symptoms and/or viral load. In some embodiments, an effective dose of the pharmaceutical composition comprising the 7DW8-5 analogue is once or multiple times daily until the subject no longer requires therapy or disease symptoms have resolved. In some embodiments, the subject can be monitored throughout the course of treatment and that the dosage of the pharmaceutical composition disclosed herein that is administered can be adjusted according to treatment progress.

In some embodiments, the subject matter described herein relates to methods of treating or preventing a respiratory infection in a subject in need thereof. In one embodiment the subject is immunocompromised. In some embodiments, the subject is infected with HIV. In some embodiments, the subject is infected with mycobacteria. In some embodiments, the subject is infected with malaria. In some embodiments, the subject is infected with HIV, mycobacteria, or malaria. In some embodiments, the subject is afflicted with cancer. In some embodiments, the subject is at an elevated risk for cancer. In some embodiments, the subject has or is at an elevated risk for an autoimmune disease. In some embodiments, the subject has an inappropriate or undesirable immune response.

The pharmaceutical composition comprising a glycolipid described herein can be formulated in any pharmaceutically acceptable carrier, excipient, or a combination thereof. In some embodiments, the glycolipid is α-GalCer. In some embodiments, the glycolipid is an α-GalCer analogue. In some embodiments, the glycolipid is 7DW8-5. Exemplary carriers or excipients include calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In some embodiments, the pharmaceutical composition described herein is administered with a pharmaceutical agent. In some embodiments, the pharmaceutical agent is an immunostimulant. In some embodiments, the pharmaceutical agent is an anti-viral agent. In some embodiments, the pharmaceutical agent is an anti-bacterial.

In some embodiments, the pharmaceutical composition described herein is administered by a medical professional. In some embodiments, the pharmaceutical composition described herein is administered by a person with no medical background. In some embodiments, the pharmaceutical composition described herein is administered by the subject receiving the pharmaceutical composition. In some embodiments, the pharmaceutical composition described herein is administered in a clinical setting. In some embodiments, the pharmaceutical composition described herein is administered in a non-clinical setting. In some embodiments, the pharmaceutical composition described herein is administered in a domestic setting.

### Subjects

In some embodiments, the “subject” described herein includes, but is not limited to, a vertebrate animal. In some embodiments, the “subject” is a mammal or a mammalian species. In some embodiment, the “subject” is a human. In some embodiments, the “subject” is a human patient. In some embodiments, the “subject” is a human child. In some embodiments, the “subject” is a human adult. In some embodiments, the “subject” is a non-human vertebrate animal, including, without limitation, non-human primates, laboratory animals, livestock, racehorses, domesticated animals, and non-domesticated animals. In some embodiments, a “mammal” includes, but is not limited to, a human, mouse, rat, guinea pig, dog, cat, horse, cow, pig, or non-human primate, such as a monkey, chimpanzee, baboon or rhesus.

In some embodiments, the subject is diagnosed with or is suspected of having a viral respiratory infection. In some embodiments, the subject is diagnosed with or is suspected of having a coronavirus infection. In some embodiments, the subject is diagnosed with or is suspected of having a SARS-COV-2 virus infection. In some embodiments, the subject is diagnosed with or is suspected of having a SARS-COV-2 variant virus infection. In some embodiments, the subject is diagnosed with or is suspected of having a SARS-COV-2 Omicron variant virus infection. In some embodiments, the subject is diagnosed with or is suspected of having a SARS-COV-2 Delta variant virus infection. In some embodiments, the subject is diagnosed with or is suspected of having an RSV virus infection. In some embodiments, the subject is diagnosed with or is suspected of having an influenza virus infection.

### REFERENCES

1. Rosendahl Huber S, van Beek J, de Jonge J, Luytjes W, van Baarle D. T cell responses to viral infections-opportunities for Peptide vaccination. Front Immunol. 2014 April; 5.

2. Fujii S-I, Shimizu K, Smith C, Bonifaz L, Steinman R M. Activation of natural killer T cells by alpha-galactosylceramide rapidly induces the full maturation of dendritic cells in vivo and thereby acts as an adjuvant for combined CD4 and CD8 T cell immunity to a coadministered protein. J Exp Med. 2003 July; 198(2): pp. 267-279.

3. Taraban V Y, Martin S, Attfield K E, Glennie M J, Elliott T, Elewaut D, Van Calenbergh S, Linclau B, Al-Shamkhani A. Invariant NKT cells promote CD8+ cytotoxic T cell responses by inducing CD70 expression on dendritic cells. J Immunol. 2008 April; 180(7): pp. 4615-4620.

4. Simara P, Tesarova L, Tapuchova I, Celerova J, Koutna I. T-Cell Activation: Post-Infection Diagnostic Tool for COVID-19. Folia Biol (Praha). 2021; 67(1): pp. 16-27.

5. Li X, Wu D, Fujio M, Imamura M, Vasan S, Wong C-H, Ho D D, Tsuji M. 2010. Discovery of a novel CD1d-binding NKT cell ligand as a vaccine adjuvant. Proc. Natl. Acad. Sci. USA. 107: 13010-13015.

6. Porcelli, S. A. & Modlin, R. L. (1999) Annu. Rev. Immunol. 17, 297-329).

### EXAMPLES

The following examples illustrate the present invention, and are set forth to aid in the understanding of the invention, and should not be construed to limit in any way the scope of the invention as defined in the statements of the invention which follow thereafter. The Examples described below are provided to illustrate aspects of the present invention and are not included for the purpose of limiting the invention.

### Example 1-7DW8-5 Stimulates NKT Cells to Activate/Mature Dendritic Cells (DSc) as Part of the Innate Immune Response

### Abstract

The fear and devastation caused by the COVID-19 pandemic has been mitigated by the successful development and deployment of prophylactic vaccines that substantially lowered the incidences of symptomatic infection, hospitalization, and death^{1,2}. However, as the causative agent SARS-COV-2 continues to spread and evolve worldwide, vaccine-breakthrough infections have become frequent, especially after the emergence of viral variants that are antigenically distant from the ancestral strain used in the current vaccines^{3,4}. Additional approaches are therefore needed in our prevention tool kit. In some embodiments, the subject matter described herein relates to a glycolipid termed 7DW8-5 that exploits the host innate immune system to enable rapid control of viral infections in vivo. This glycolipid binds to CD1d on antigen-presenting cells and thereby stimulates NKT cells to release a cascade of cytokines and chemokines. The intranasal administration of 7DW8-5 prior to virus exposure significantly blocked infection by three different variants of SARS-COV-2, as well as by respiratory syncytial virus and influenza virus, in mice or hamsters. This protective antiviral effect is both host-directed and mechanism-specific, requiring both the CD1d molecule and interferon-γ. A chemical compound like 7DW8-5 that is easy to administer and cheap to manufacture may be useful not only in slowing the spread of COVID-19 but also in responding to future pandemics due to a respiratory virus long before vaccines or drugs are developed.

### Introduction

The COVID-19 pandemic has devastated humanity, but the scientific response to develop countermeasures against the causative agent, SARS-COV-2, has been truly unprecedented. In record time, antiviral drugs^{6,7} and monoclonal antibodies^{8,9} were added to the therapeutic arsenal. Likewise, many prophylactic vaccines were developed to harness the power of the adaptive immune response, with several shown to be highly protective against symptomatic infection^{1,2}. However, as SARS-COV-2 continued to spread and new viral variants continued to evolve, vaccine-breakthrough infections have become a common occurrence, particularly after the emergence of the Omicron subvariants that are antigenically most distinct from ancestral strains^{3,4}. Novel prevention strategies could help to slow the transmission of this viral pathogen worldwide, including approaches that exploit the innate immune system and enable rapid control of infection.

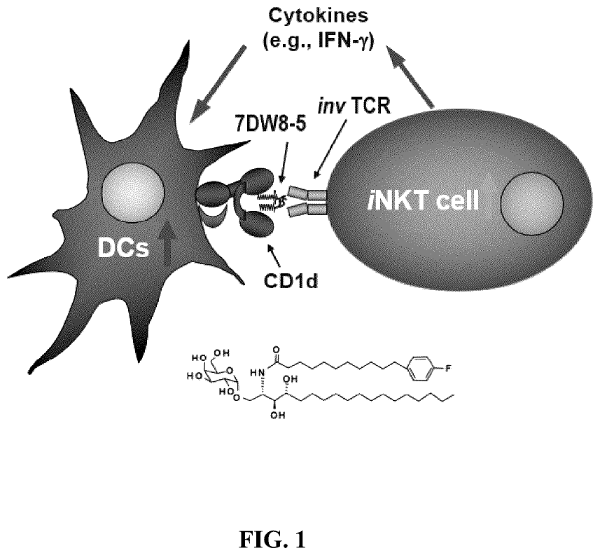

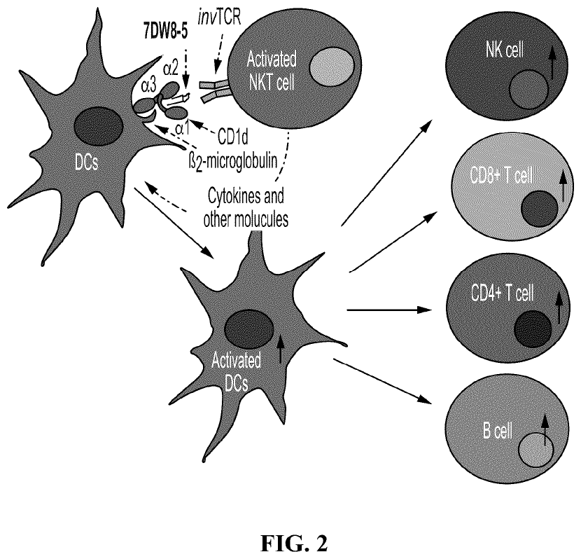

Natural killer T (NKT) cells are a subset of lymphocytes possessing features of both natural killer (NK) cells and αβ T cells^{10,11}. These cells form a key element of the innate immune response and may play a role not only in cancer¹²⁻¹⁴ and autoimmune diseases¹⁵, but also in protection against infections¹⁶⁻¹⁸. Some NKT cells possess a semi-invariant T-cell receptor (iTCR) and are thus called invariant NKT cells (INKT cells), which recognize certain glycolipids bound to CD1d molecules on antigen-presenting cells (e.g., dendritic cells or DCs) and thereby trigger an immune cascade^{10,11} (FIG. 1). The first CD1d ligand identified was a glycolipid termed α-galactosylceramide (α-GalCer)²⁰. From a focused library of synthetic glycolipids, an α-GalCer analogue has since been discovered, 7DW8-5 (FIG. 1), that exhibits even more potent immunostimulatory activity in both mouse and human iNKT cells in vitro⁵. Upon stimulation, iNKT cells can not only secrete Th1 cytokines that are known to have antiviral effects²¹⁻²³, but can also activate populations of NK cells and CD8+ T cells^{14,24,25}. Each of these activated cell populations can release multiple cytokines, including interferon-γ (IFN-γ) that has a protective effect against several viral infections²⁶⁻²⁸. In some embodiments, the subject matter described herein relates to the mechanism-specific effect of 7DW8-5 in preventing infection by SARS-COV-2, respiratory syncytial virus (RSV) and influenza virus in animal models. FIG. 2 shows a schematic representation of the mode of NKT cell activation by the 7DW8-5 glycolipid and subsequent activation of various immune competent cells. Activated DCs in turn activate a number of cells involved in the innate immune response and the non-specific clearance of pathogens, such as NK cells, CD8+ T cells, CD4+ T cells, and B cells.

### 7DW8-5 Blocks SARS-COV-2 Infection

Without being bound by theory, the immunostimulatory effect of 7DW8-5 inhibits SARS-COV-2 infection in mice. 7DW8-5 (2 μg) was administered intravenously (IV) or intranasally (IN) to groups of BALB/c mice two days before each animal was challenged IN with 5×10⁴ plaque-forming units (PFU) of the mouse-adapted MA10 strain²⁹ of SARS-COV-2. A third group of mice received saline and served as controls, while a fourth group was given 7DW8-5 (2 μg; IN) at 2 hours post virus challenge.

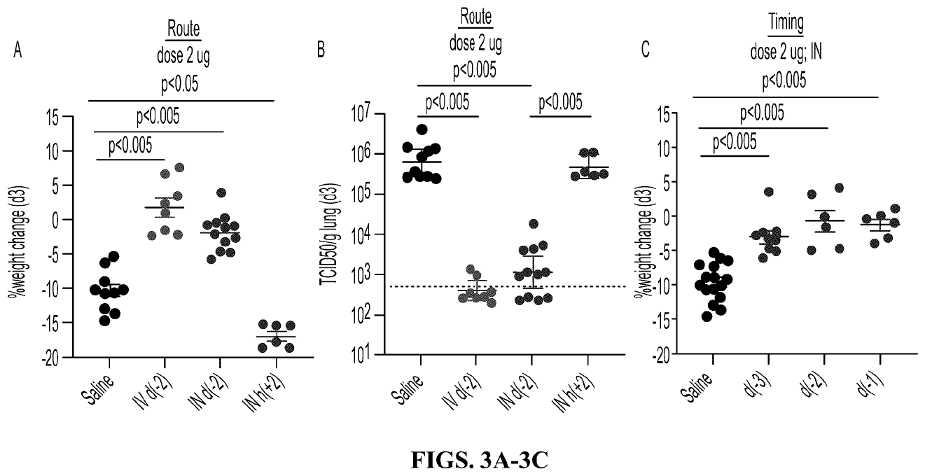

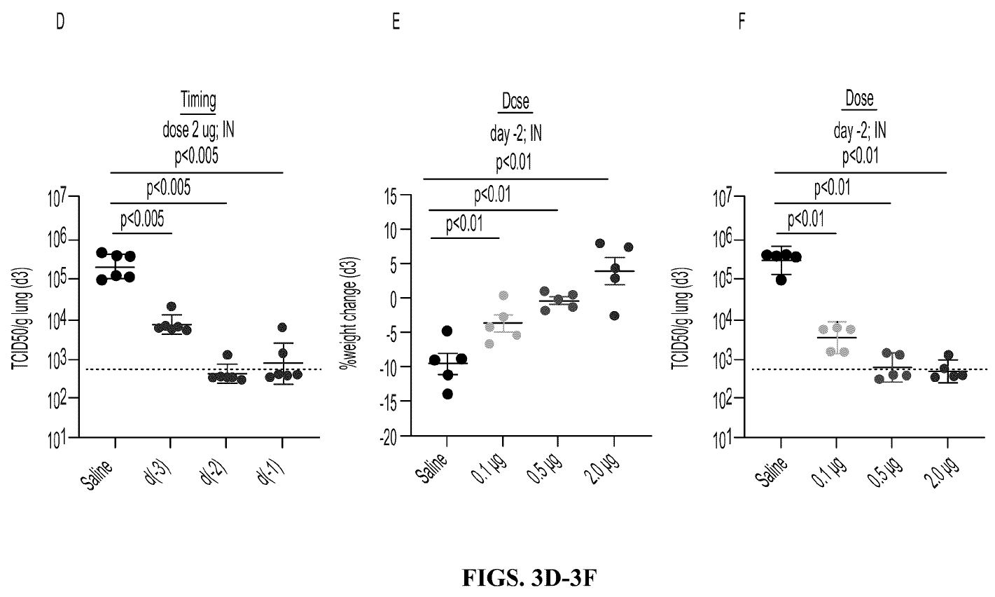

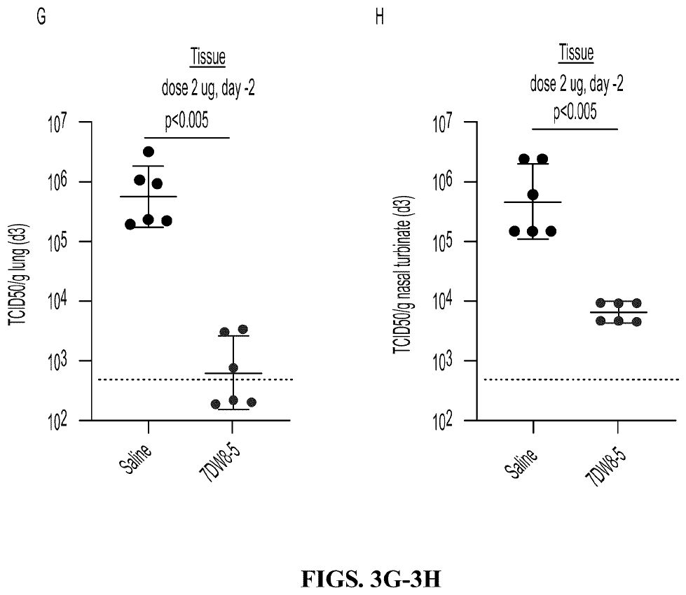

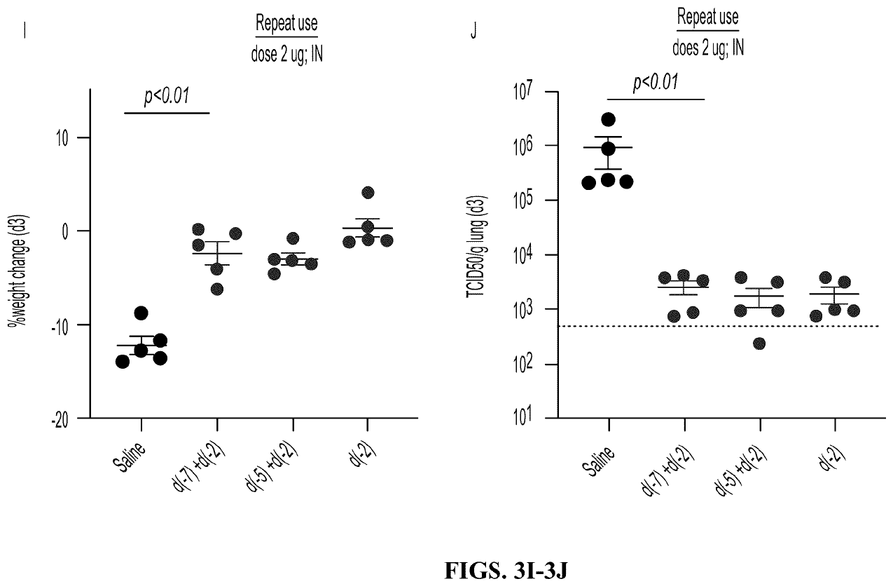

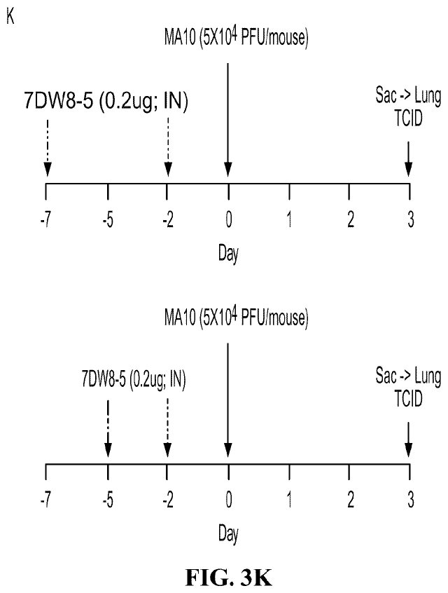

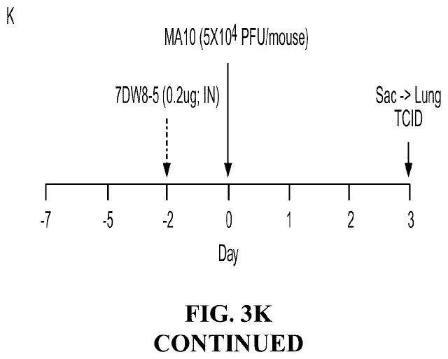

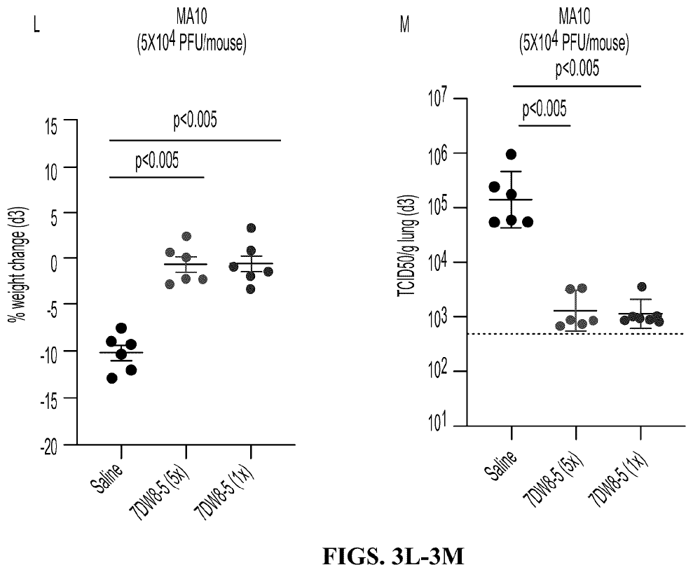

7DW8-5 administration protects mice from a viral infection when administered in a dosage of 2 μg intravenously or intranasally one day before SARS-COV-2 MA10 exposure. Fourteen-15 weeks old BALB/c mice (N=6-12) were given 2 μg of 7DW8-5 by intravenous (IV) or intranasal (IN) route one day before challenge with 50,000 pfu of SARS-COV-2 MA10 (mouse-adapted) strain (by IN). Two μg of 7DW8-5 was given by IN route also 2 h post SARS-COV-2 challenge. Three days after SARS-COV-2 challenge, lungs were harvested from mice and homogenized, and the supernatants were collected and the viral load was determined by 50% tissue culture infectious dose (TCID₅₀) endpoint dilution assay, in which the supernatants were serially diluted onto Vero E6 cells and viral titer (by CPE) measured 70 h later. When compared to controls, both IV and IN pre-exposure administrations of 7DW8-5 resulted in significant protection against MA10 infection as manifested by preservation of body weight and striking reductions of infectious virus in lung tissues (FIGS. 3A-B). In contrast, the post-exposure administration of 7DW8-5 failed to show any protective effect. A follow-up study was performed to assess the duration of the prophylactic effect of 7DW8-5 (2 μg; IN). Strong protection against MA10 infection was observed when this glycolipid was given to mice either one or two days prior to virus exposure, whereas the protection was only partial when given three days prior (FIGS. 3C-D). A dose-exploration experiment then demonstrated robust protection of 7DW8-5 (IN; 2 days prior) at doses of 0.5 μg or 2 μg, whereas the protection was suboptimal at a dose of 0.1 μg (FIGS. 3E-F).

To evaluate whether the antiviral effect of the glycolipid could be seen at the site of virus inoculation, mice were again given 7DW8-5 (2 μg; IN) two days before MA10 challenge, followed by harvesting of nasal turbinates and lungs three days later. Marked reductions of SARS-COV-2 replication were observed in both sets of tissues from mice treated with 7DW8-5 (FIGS. 3G-H). FIG. 3I shows that infected mice treated with 7DW8-5 using all administration schedules have higher body weight than those treated with saline alone. FIG. 3J shows that infected mice treated with 7DW8-5 using all administration schedules have lower virus titer than those treated with saline alone. FIG. 3K shows a schematic representation of the administration schedules in FIGS. 3I-J. Body weight was measured for each mouse prior to SARS-COV-2 infection and on day 3 post infection. Next, it was assessed whether the repeated dosing of 7DW8-5 could lead to possible NKT-cell energy and thus the loss of its antiviral efficacy. To this end, 7DW8-5 (0.2 μg; IN) was administered to a group of mice every other day for 10 days before MA10 challenge. Serving as a comparator, another group of mice received the same glycolipid (0.2 μg; IN) only once at two days prior to virus exposure. The observed protective effects were equivalent for the two groups (FIGS. 3L-M), indicating lack of energy with repeated dosing of 7DW8-5 under these experimental conditions.

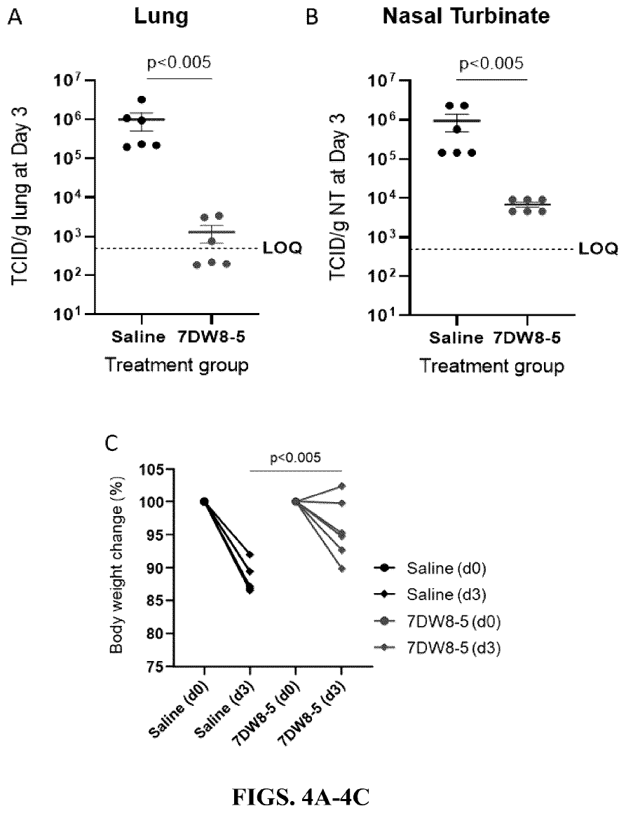

FIGS. 4A-C show that the prophylactic activity of 7DW8-5 against MA10 is observed in the lungs and the nasal turbinate of mice infected mice. FIG. 4A shows viral titer in lings. FIG. 4B shows viral titer in the nasal turbinate. FIG. 4C shows body weight change in mice.

7DW8-5 has protective effects against the Omicron and Delta variants of SARS-CoV-2. Fourteen-15 weeks old BALB/c mice (N=6) were given 2 μg of 7DW8-5 by IN route one day before challenge with 150,000 pfu of SARS-COV-2 Omicron strain (by IN). Three days after SARS-COV-2 challenge, lungs were harvested from mice and homogenized, and the supernatants were collected and the viral load was determined by TCID₅₀ assay, in which the supernatants were serially diluted onto Vero E6 cells and viral titer (by CPE) measured 70 h later. Similar experiments were also performed with hamsters.

### Breadth of Protection

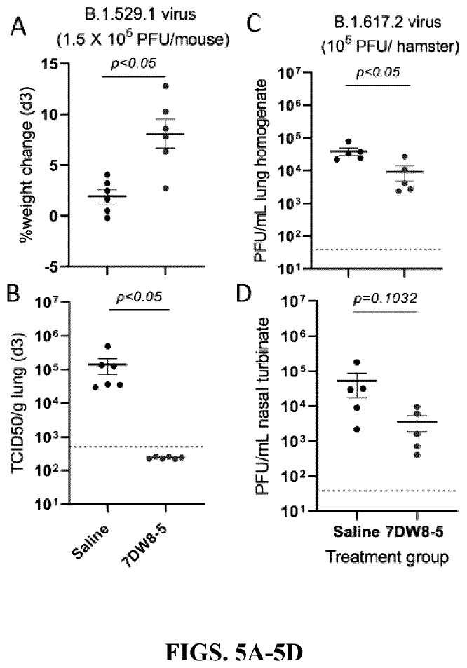

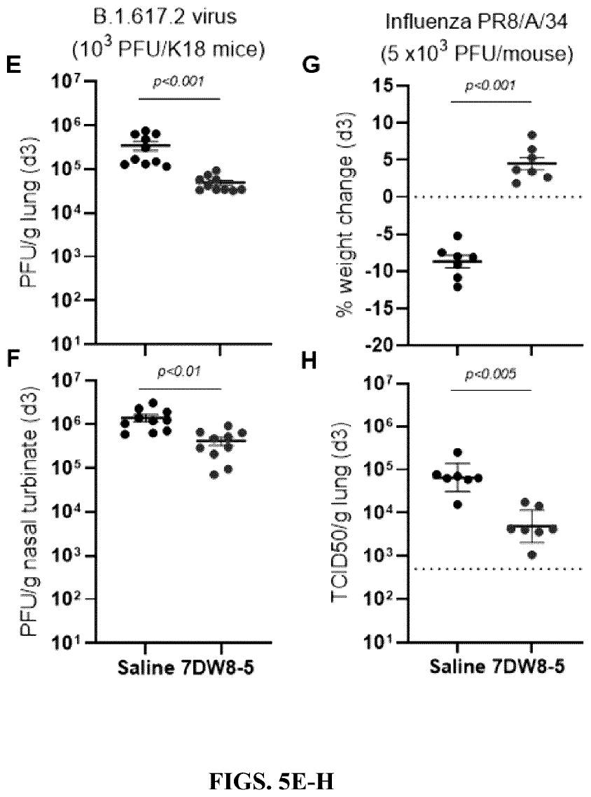

The antiviral effect of 7DW8-5 against other SARS-COV-2 strains was also assessed, starting with the Omicron BA. 1 variant (B.1.1.529), which had been shown to infect wild-type mice without prior adaptation. However, this infection model typically resulted in no weight loss in the infected animals as well as lower levels of virus replication³⁰. Compared to controls, administration of 7DW8-5 (2 μg; IN) 2 days before the virus challenge (1.5×10⁵ PFU) led to significant weight gain and complete inhibition of virus replication in lung tissues (FIGS. 5A-B). 7DW8-5 (100 μg/kg; IN) given 2 days before Delta variant challenge (105 PFU) significantly decreased the viral load in lung and nasal tissues of treated hamsters (FIGS. 5C-D). A similar experiment was carried out using the Delta variant (B.1.617.2), which cannot infect wild-type mice but can infect K18-human ACE2-transgenic mice as well as hamsters³¹. Once more, administration of 7DW8-5 (2 μg; IN; 2 days prior) significantly lowered the viral load in both lung and nasal tissues of the virus-challenged (10³ PFU) transgenic mice (FIGS. 5E-F).

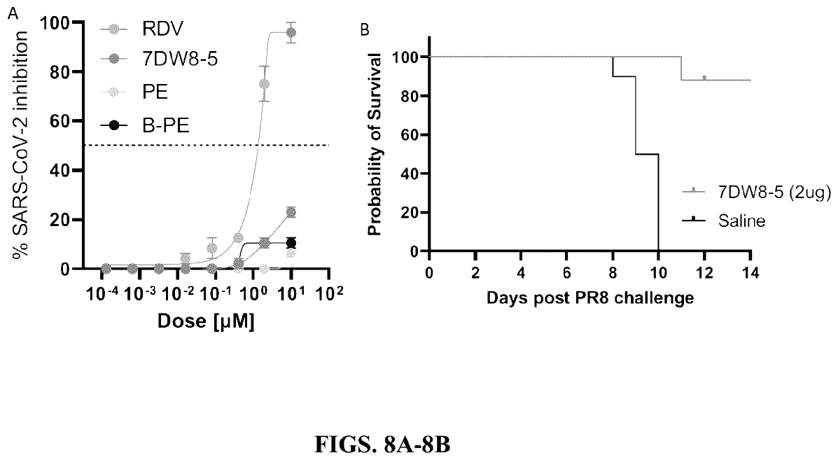

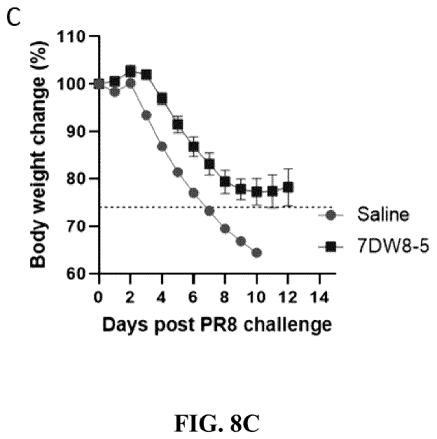

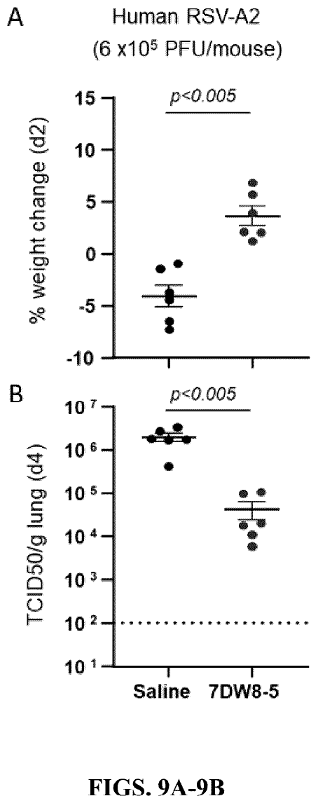

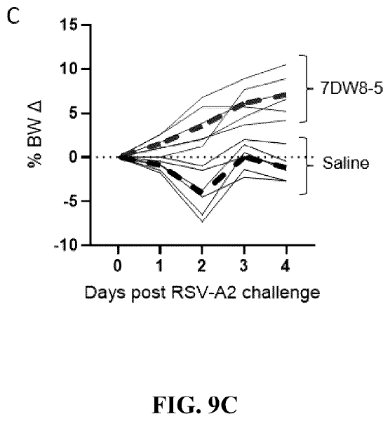

Given that 7DW8-5 had no direct antiviral activity against SARS-COV-2 in vitro (FIG. 8A) and was likely stimulating a part of the innate immune system, it was then explored whether its protection could be extended to other clinically important viral pathogens, including RSV and influenza virus. For the former, the RSV-A2 strain known to infect BALB/c mice was used³². As shown in FIGS. 9A-B, administration of 7DW8-5 (2 μg; IN; 2 days prior) significantly prevented weight loss and inhibited virus replication in lung tissues of treated mice. For the influenza studies, strain A/H1N1/PR8 known to infect C57BL/6 mice was used³³. Again, administration of 7DW8-5 (2 μg; IN; 2 days prior) significantly maintained body weight as well as reduced the amount of infectious virus in lung tissues of treated mice (FIGS. 5G-H). In a longer-term experiment using a lethal dose (5×10³ PFU) of influenza A/H1N1/PR8, all control mice died by Day 10 after virus exposure as expected, whereas 8 of 10 mice given 7DW8-5 survived (FIG. 8B). Taken together, these findings suggest that the protective effect of this glycolipid has substantial breadth against divergent respiratory viruses, perhaps due to stimulation of a specific immunologic pathway.

### Mechanism of Protection

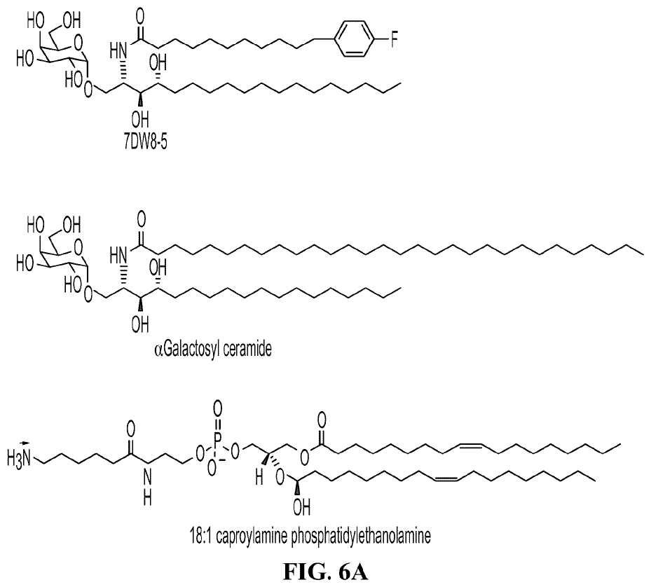

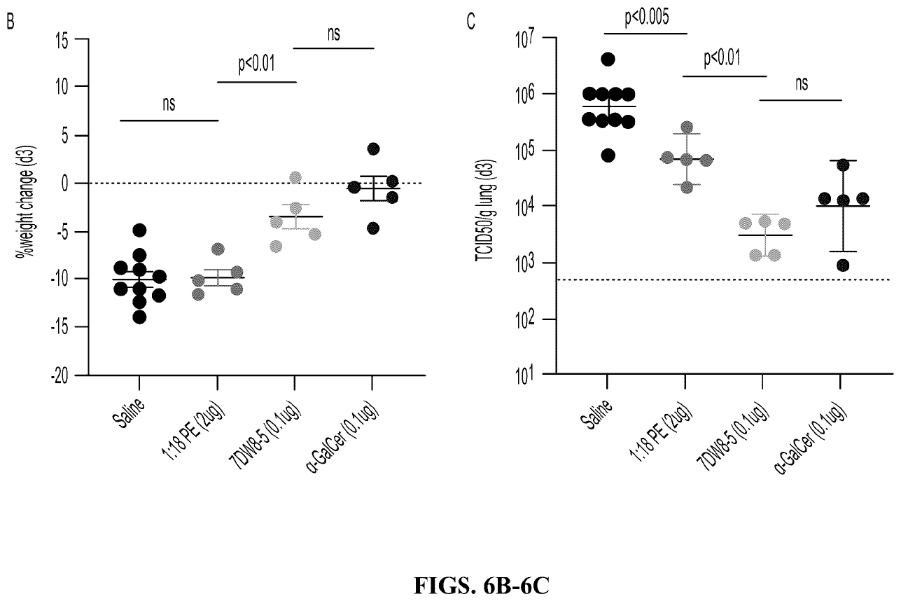

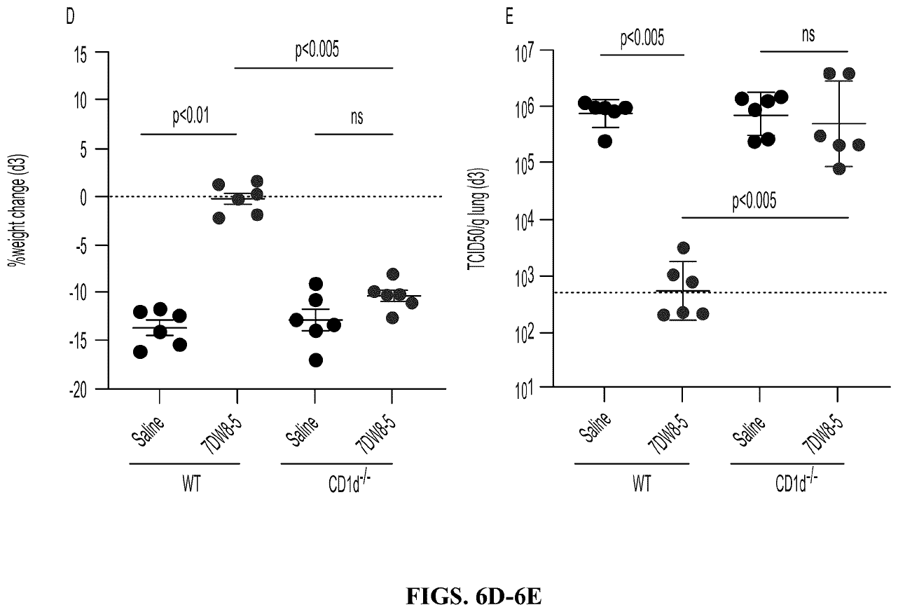

Next, the mechanism of protection for 7DW8-5 was addressed. First, the role of CD1d-mediated activation was examined by comparing 7DW8-5 to its active parental compound, α-GalCer, and to 1:18 Caproylamine PE, which is known to bind to CD1d without stimulating iNKT cells due to the lack of a sugar moiety (FIG. 6A)^{5,36}. Both 7DW8-5 and α-GalCer administration (0.1 μg; IN; 2 days prior) resulted in protection, as manifested by maintenance of body weight and reduction of viral replication in lung tissues of mice challenged with the MA10 strain of SARS-COV-2 (FIGS. 6B-C). In contrast, administration of 1:18 Caproylamine PE at a much higher dose (2 μg; IN) failed to protect. This finding led, logically, to a subsequent experiment using CD1d knock-out (KO) mice. Indeed, the protective effect of 7DW8-5 observed in wild-type mice against SARS-COV-2 MA10 challenge was completely abolished in CD1d-KO mice (FIGS. 6D-E), demonstrating the indispensable role of CD1d activation in the antiviral activity of 7DW8-5.

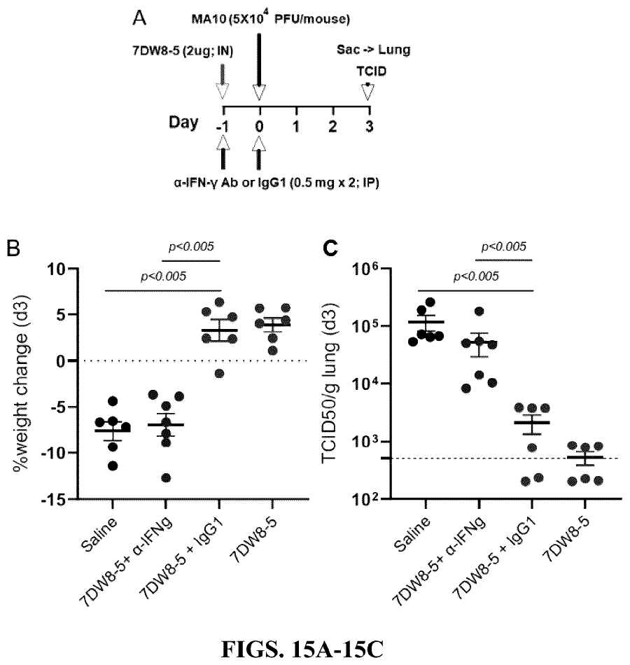

The role IFN-γ played in contributing to the biological activity of 7DW8-5 was determined by conducting protection studies in mice deficient in IFN-γ. The prophylactic effect of 7DW8-5 against MA10 is primarily mediated by IFN-γ as shown in FIGS. 15A-C. The anti-SARS-COV-2 effect of 7DW8-5 in wild-type mice was largely eliminated by the intraperitoneal injection of an anti-mouse-IFN-γ monoclonal antibody prior to the glycolipid treatment (FIGS. 15B-C). There were six to seven mice per treatment condition group. FIG. 15A shows a schematic representation of experimental set-up and analysis. One day before virus exposure, mice were administered saline, 7DW8-5 alone, or 7DW8-5 administered with an anti-IFN-γ antibody or a control antibody. Tissue was harvested at 3 days post-infection. FIG. 15B shows that mice treated with 7DW8-5 alone or with a control antibody (does not target IFN-γ) have higher body weight than mice treated with saline or 7DW8-5 and anti-IFN-γ antibody. These results suggest that the effects of 7DW8-5 on body weight in mice are mediated through IFN-γ. Mice treated with 7DW8-5 alone or with 7DW8-5 and a control antibody have larger body weight change than those treated with saline or 7DW8-5 and an anti-IFN-γ antibody. FIG. 15C shows that mice treated with 7DW8-5 alone or with a control antibody (does not target IFN-γ) have lower virus titer that mice treated with saline or 7DW8-5 and anti-IFN-γ antibody. These results suggest that the effects of 7DW8-5 on virus titer in mice are mediated through IFN-γ. Virus exposure consisted of 50,000 PFUs of MA10. The dose of was 7DW8-5 2 μg IN on day −1 prior to virus exposure. The antibody dose was 0.5 mg administered intraperitoneally (IP) on days −1 & 0. The antiviral activity of 7DW8-5 observed in wild-type mice against SARS-COV-2 MA10 challenge was totally lost in IFN-γ-KO mice (FIGS. 15D-E). These latter results prove that IFN-γ is another essential mediator of the activity of 7DW8-5, along with CD1d.

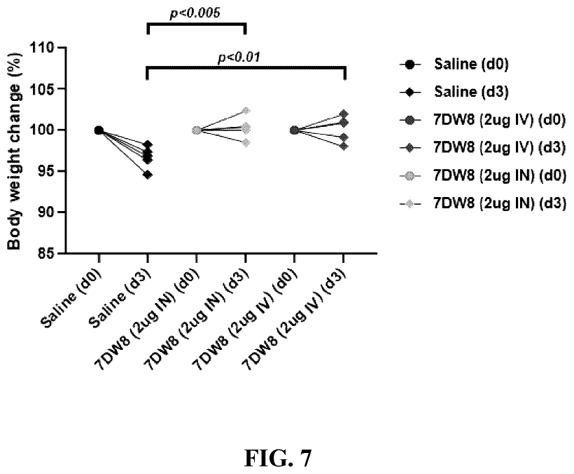

FIG. 7 shows lack of body weight loss in 7DW8-5-treated C57BL/6 mice challenged with influenza A virus PR8 strain. Fourteen-15 weeks old C57BL/6 mice (N=5) were given 2 μg of 7DW8-5 by IN route one day before challenge with 200 pfu of influenza A virus (H1N1) PR/8/34 (PR8) strain (by IN). Body weight was monitored for each mouse prior to challenge and on day 3 post infection (end of experiment).

FIGS. 8A-B show that 7DW8-5 is not an anti-SARS-COV-2 compound and that it blocks infection with influenza virus (PR8). Fourteen-15 weeks old C57BL/6 mice (N=10) were given 2 μg of 7DW8-5 by IN route one day before challenge with 5000 pfu of influenza A virus PR8 strain (by IN). FIG. 8A shows the lack of a direct antiviral effect against SARS-CoV-2 in vitro with 7DW8-5. The positive control remdesivir is a nucleotide prodrug of an adenosine analog. It binds to the viral RNA-dependent RNA polymerase and inhibits viral replication by terminating RNA transcription prematurely. Remdesivir exhibits activity against the SARS-COV-2 virus. FIG. 8B shows higher probability of survival of mice infected with influenza and treated 7DW8-5 than those treated with saline. FIG. 8C shows that body weight loss is ameliorated in treated mice with 7DW8-5. Body weight and mortality were monitored for each mouse prior to challenge and every single day after the challenge.

The data disclosed herein shows that 7DW8-5 protects against SARS-COV-2 MA10 infection in mice at 2 μg dose given intranasally or intravenously 1-3 day prior to exposure to the virus. However, no protection is observed when 7DW8-5 is administered 2 h post virus challenge. Moreover, the protective effects of 7DW8-5 are dose dependent. It was observed that 7DW8-5 strongly protects against SARS-COV-2 MA10 infection at 0.5 μg dose and partially protects at 0.1 μg dose in mice. As shown, the antiviral effects of 7DW8-5 are mediated through CD1d and iNKT cell stimulation because 1:18 phosphoethanolamine (PE), which binds CD1d but lacks iNKT cell stimulation, fails to protect mice against a SARS-COV-2 MA10 infection.

7DW8-5 does not protect against SARS-COV-2 MA10 in CD1d-deficient mice. The protective effects of 7DW8-5 against SARS-COV-2 MA10 were seen not only in the lungs, but also in the nasal turbinates of the mice, bony structures inside the nose covered by mucosa. Additionally, 7DW8-5 protects mice against the SARS-COV-2 Omicron as well as an influenza (PR8) exposure.

### Example 2—RSV Inhibition With 7DW8-5 Administration in Mice

7DW8-5 clearly inhibits respiratory syncytial virus (RSV) in mice as show in FIGS. 9A-C. BALB/c mice were treated with saline or 7DW8-5 in groups of six mice per treatment condition. 7DW8-5 was administered in a dosage of 2 μg per mouse. Lung tissue was harvested on day 4 following the varus challenge to determine median tissue culture infectious dose (TCID₅₀) of the lung titer of RSV. The TCID₅₀ was determined in in Hep2 cells. FIG. 9A shows that mice infected with human RSV-2 vital strain and treated with 7DW8-5 have a higher body weight than mice infected with human RSV-2 vital strain and treated with saline. Body weight was measured on day two following the virus challenge. FIG. 9B shows that viral titer TCID/g in the lungs of infected mice is decreased with 7DW8-5 treatment. FIG. 9C shows body weight changes of individual mice. At four days following the virus challenge, all infected mice treated with 7DW8-5 have higher body weight than those treated with saline.

### Example 3—Prophylactic Activity of 7DW8-5

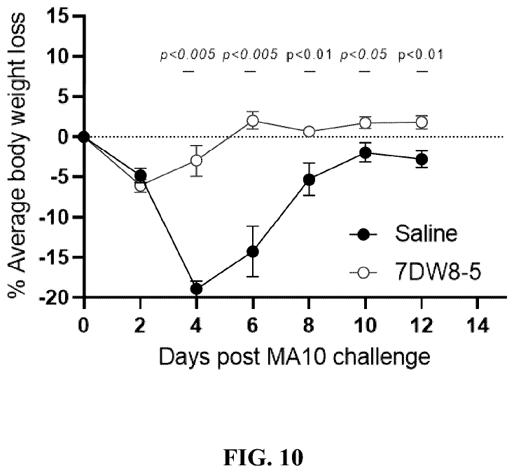

The long-term antiviral effect of 7DW8-5 administered against a MA10 challenge on mouse body weight as shown in FIG. 10. The 7DW8-5 glycolipid was administered two days prior the virus challenge (−2-day treatment). The 7DW8-5 glycolipid was administered in a dosage of 2 μg per mouse with five mice in each treatment condition group.

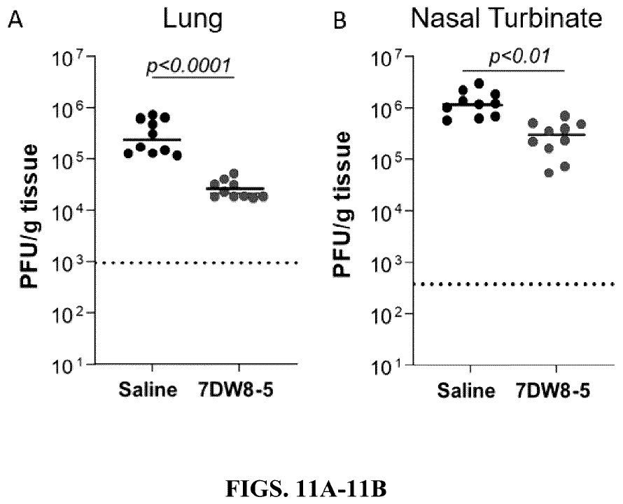

The prophylactic activity of 7DW8-5 in K18-hACE2 Tg mice against the SARS-CoV-2 B.1.617.2 (Delta) variant challenge is shown in FIG. 11A-B. The 7DW8-5 glycolipid was administered two days prior the virus challenge (-2-day treatment). The 7DW8-5 glycolipid was administered in a dosage of 2 μg per mouse with 10 mice in each treatment condition group. The virus challenge was 1000 pfu per mouse. FIG. 11A shows that the PFU per gram of lung tissue decreases with 7DW8-5 administration. FIG. 11B shows that the PFU per gram of nasal turbine tissue decreases with 7DW8-5 administration.

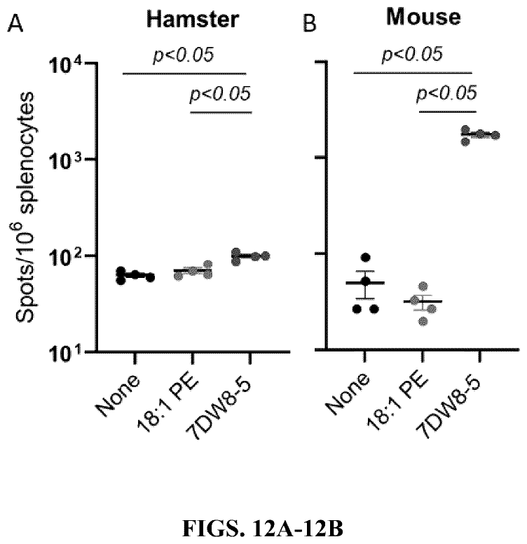

The relative number of IFN-γ-secreting splenocytes in hamster versus mouse in response to glycolipids in culture are shown in FIGS. 12A-B. 7DW8-5 administration affects IFN-γ-secreting splenocyte numbers in hamsters as shown in FIG. 12A. 7DW8-5 administration increases IFN-γ-secreting splenocytes in mice as shown in FIG. 12B. Cytokine induction

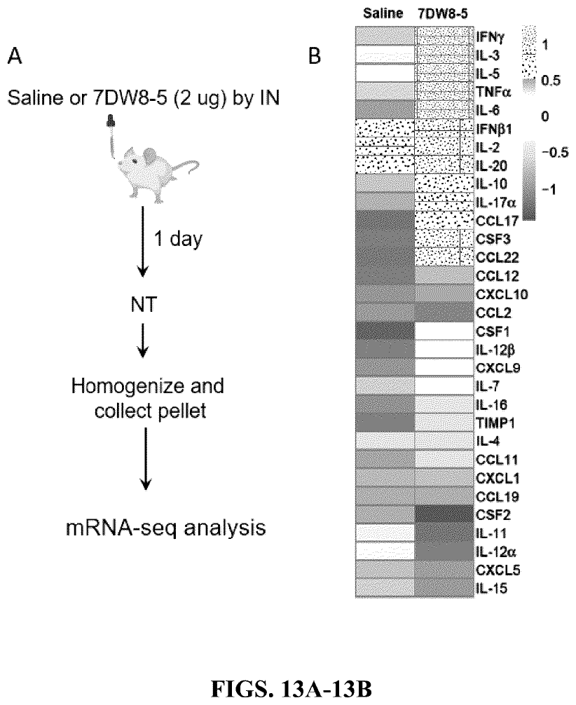

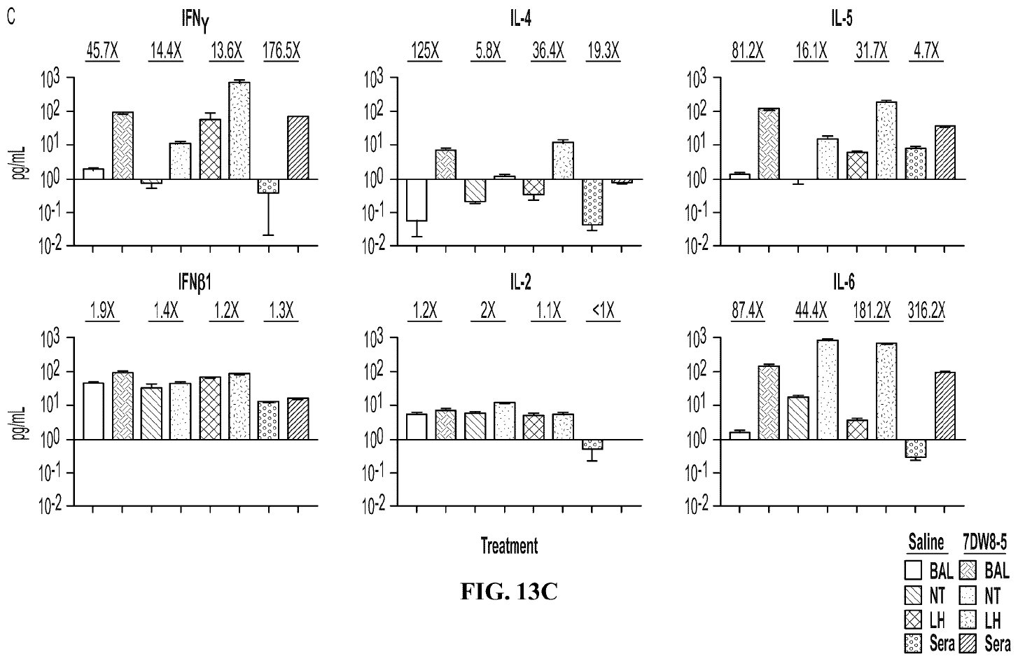

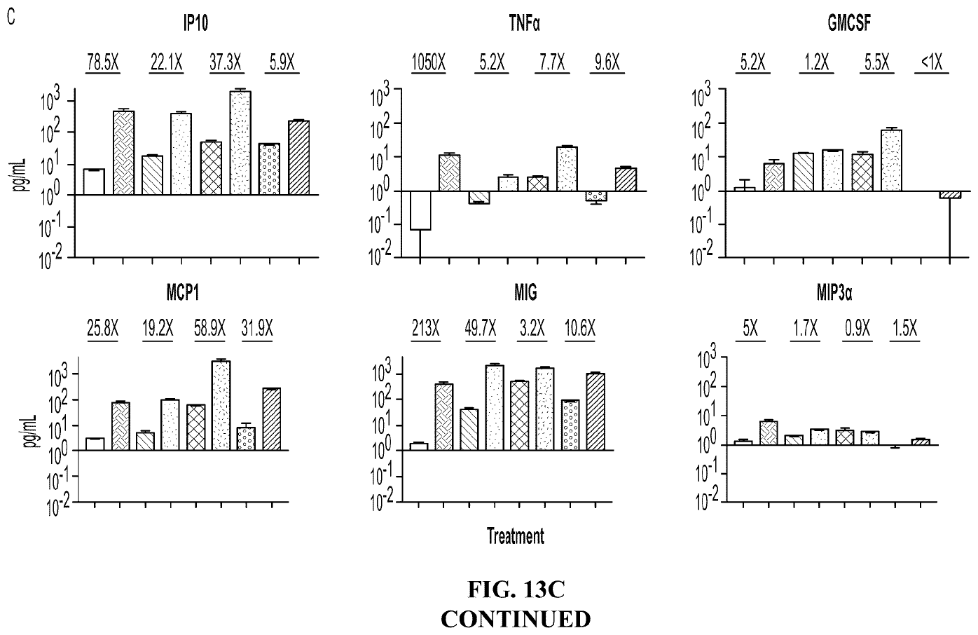

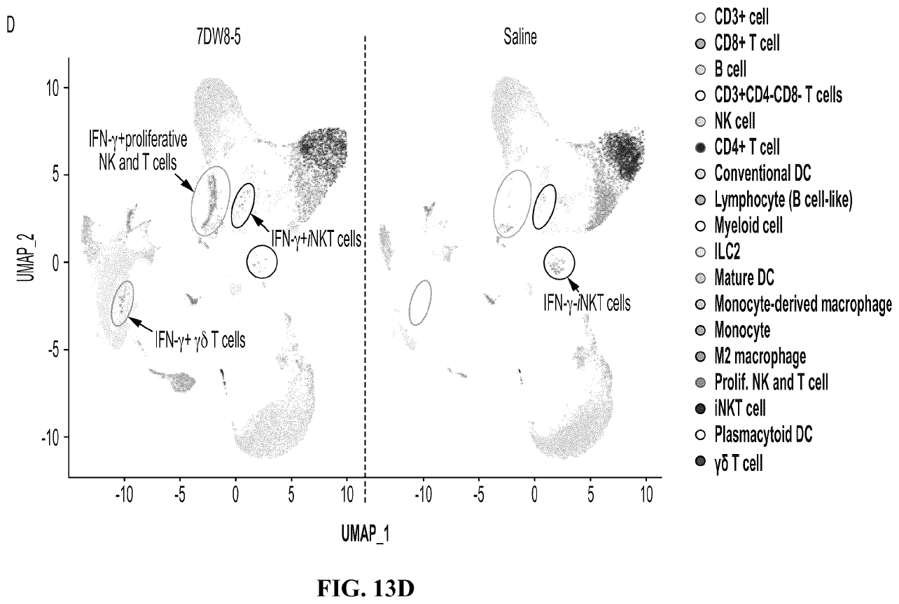

The differential expression of genes in nasal turbinate of mice upon 7DW8-5 intranasal administration is shown in FIGS. 13A-C. FIG. 13A shows a schematic representation of the mRNA-seq analysis. FIG. 13B shows RNA expression in nasal turbinate 24 hr following 7DW8-5 intranasal administration.

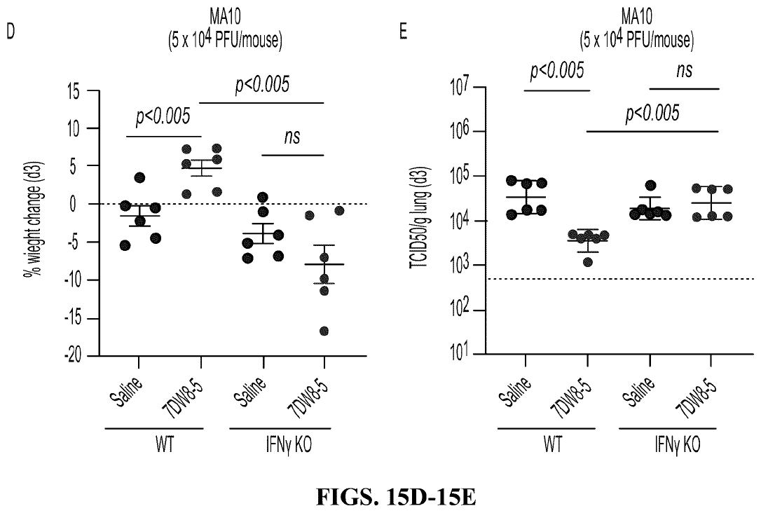

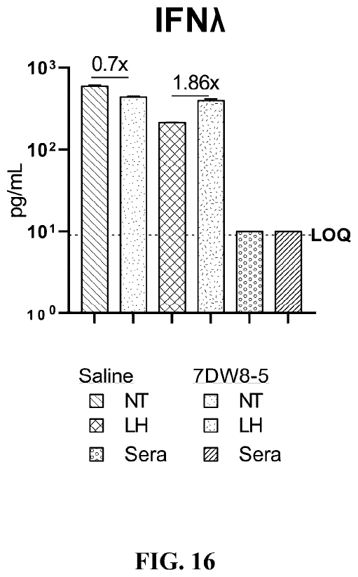

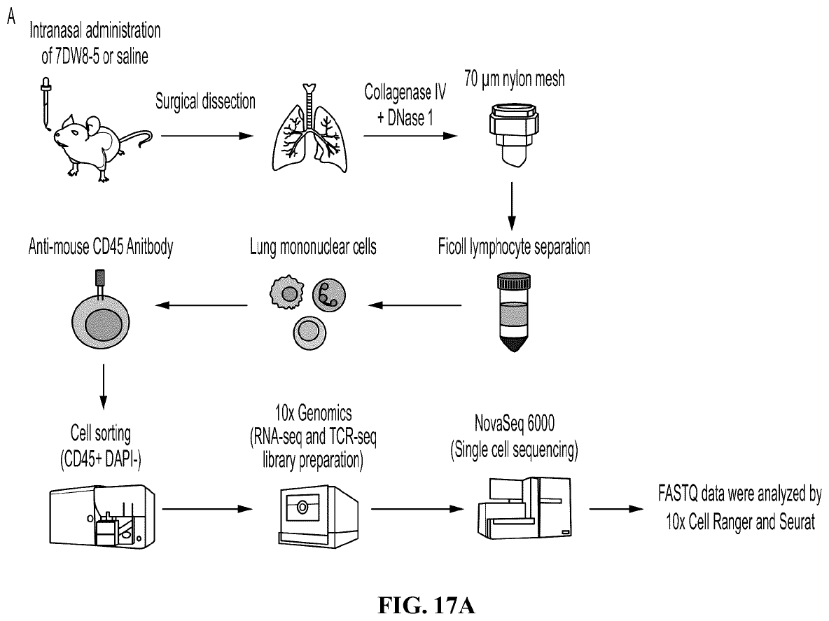

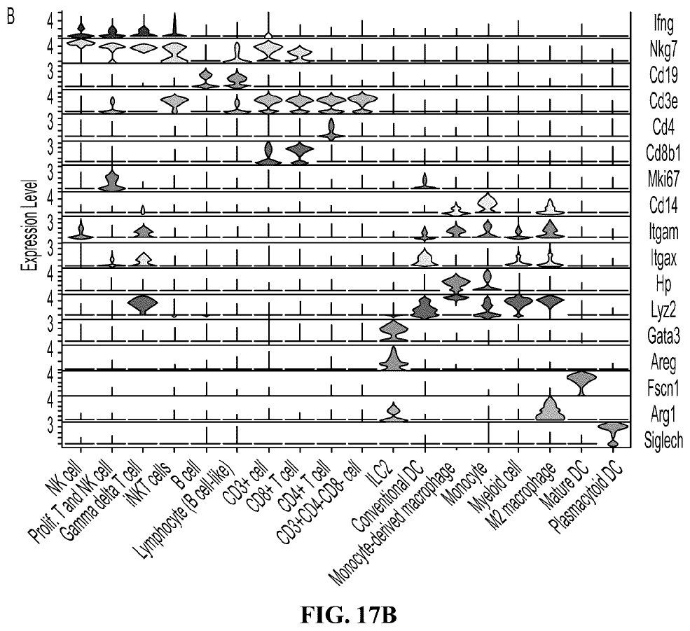

Subsequently the cytokine/chemokine profile was characterized in bronchoalveolar lavage (BAL), homogenates of nasal turbinates or lungs, and sera in BALB/c mice (N=6) one day after administration of 7DW8-5 (2 μg; IN) or saline. Marked upregulation of multiple cytokines/chemokines was detected in all compartments examined from the treated animals, including IFN-γ, IL-5, IL-6, IP-10, TNF-α, MCP1, and MIG (FIG. 13C). IFN-γ, in particular, is a known antiviral cytokine. Since IFN-γ has also been reported to have activity against SARS-COV-2 in mice³⁷, the levels of this cytokine were also measured in the supernatants of nasal turbinate and lung homogenates, as well as in sera, from mice treated with 7DW8-5 (2 μg; IN; 1 day prior) or saline. No evidence of IFN-γ induction was noted (FIG. 16). A single-cell, gene-expression analysis on lung mononuclear cells from 7DW8-5-treated mice was conducted according to a specific experimental protocol (FIG. 17A), and the expression levels of representative marker genes were documented within 18 distinct immune cell populations (FIG. 17B). The most striking observation is the apparent shift toward IFN-γ production seen in proliferative NK cells and T cells, as well as in iNKT cells and ad T cells, after exposure to 7DW8-5 (FIG. 13D). These findings implicate IFN-γ as possibly having a central role in mediating the antiviral activity of our glycolipid.

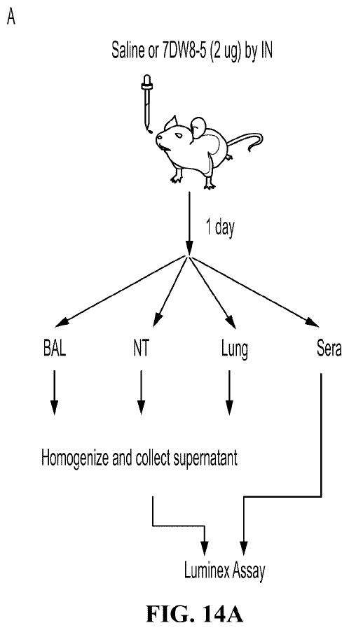

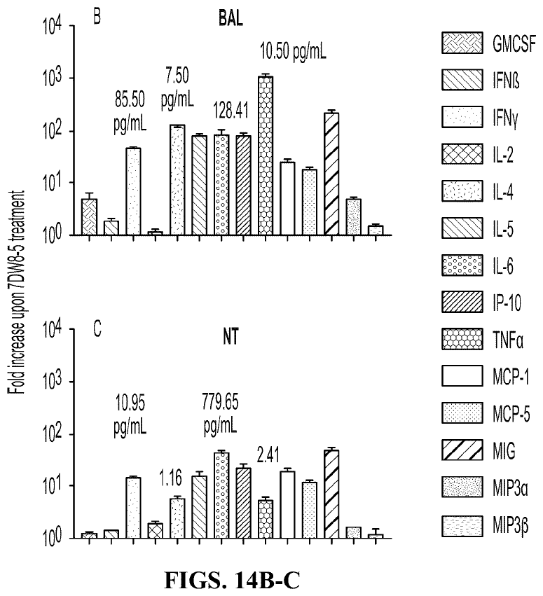

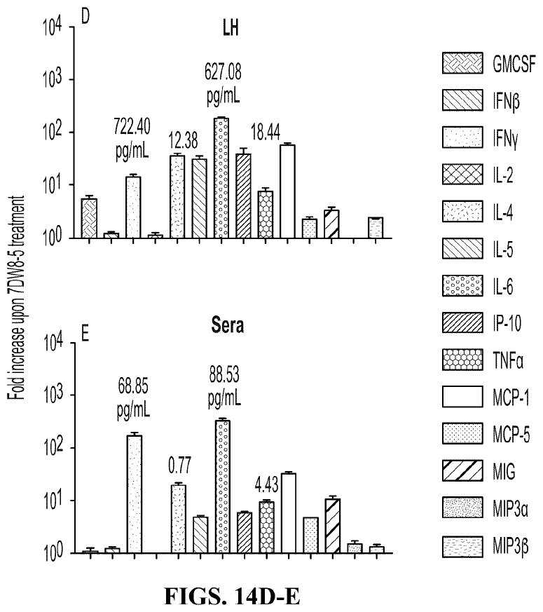

A list of cytokines/chemokines increased in mice upon 7DW8-5 intranasal administration is shown in FIGS. 14A-E. FIG. 14A shows a schematic representation of experimental set-up and analysis. FIG. 14B shows an increase in cytokines/chemokines expression in BAL. FIG. 14C shows an increase in cytokines/chemokines expression in nasal turbinate. FIG. 14D shows an increase in cytokines/chemokines expression in lung. FIG. 14E shows an increase in cytokines/chemokines expression in serum.

### Discussion

In some embodiments, intranasal administration of 7DW8-5, an immunostimulatory glycolipid, worked well as pre-exposure prophylaxis in blocking SARS-CoV-2 infection in wild-type mice, as shown by maintenance of body weight and >2 log reduction of virus replication in lung tissues (FIGS. 3A-M). The viral load in the nasal turbinates was also reduced by ˜50-fold (FIGS. 3G-H), which is more substantial than the blockade seen with monoclonal antibodies in this tissue compartment³⁷. However, post-exposure administration of 7DW8-5 was ineffective (FIGS. 3A-B), suggesting that this glycolipid will not be useful as a therapeutic. The protection conferred by 7DW8-5 extended to two SARS-COV-2 variants, including Omicron BA.1 in wild-type mice (FIG. 5A-B) and Delta in K18 transgenic mice and hamsters (FIGS. 5C-D and E-F). Moreover, comparable antiviral activity was observed in mice following challenges with RSV (FIG. 9A-b) or influenza virus (FIGS. 5G-H). These findings demonstrated the breadth of protection of 7DW8-5 versus three families of viruses (coronaviruses, paramyxoviruses, and orthomyxoviruses), each of which is not only clinically important but also of pandemic potential.

The action of 7DW8-5 is both host-directed and mechanism-specific. While many cytokines/chemokines were induced by this glycolipid (FIG. 13C), its antiviral effect was entirely abolished in CD1d-KO mice (FIGS. D-E), which was not surprising since 7DW8-5 was selected for optimal binding to and triggering through both human and mouse CD1d (FIG. 1) 5. Similarly, another essential component of the protective effect was proven to be IFN-γ, a molecule in the innate immune system known to possess broad antimicrobial activities34. 7DW8-5 showed no protection at all in IFN-γ-KO mice (FIGS. 15D-E), and its protective effect in wild-type mice was largely lost when pre-treatment with an anti-IFN-γ monoclonal antibody was given (FIGS. 15B-C). Therefore, it has demonstrated that both CD1d and IFN-γ are necessary for the activity of 7DW8-5 in vivo.

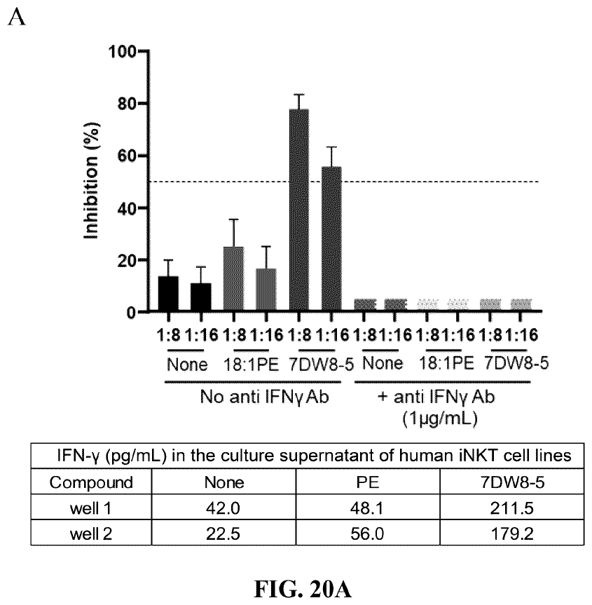

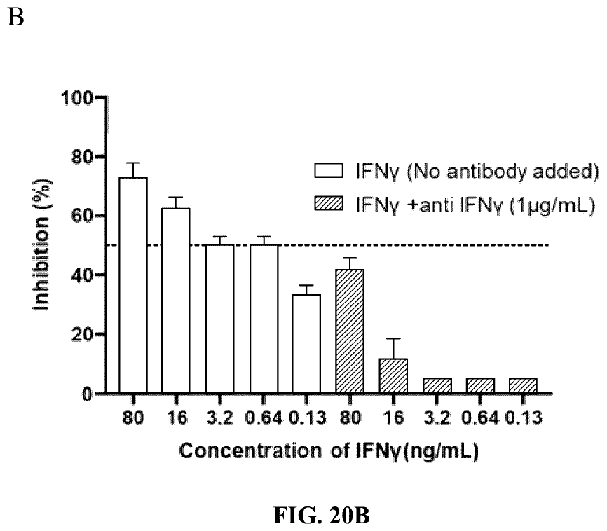

Without being bound by theory, 7DW8-5 can be an agent for pre-exposure prophylaxis to prevent infections by SARS-COV-2, RSV, influenza virus, and possibly other clinically important respiratory viruses. It is a chemical compound that is thermostable and thus simple to ship and store, cheap to manufacture, and easy to administer intranasally. It is expected that 7DW8-5 is similarly effective in people. That is because, first, this glycolipid was selected from a library of analogues based on its potent stimulatory effect on human INKT cells in vitro⁵. Second, iNKT cells constitute up to 1% of peripheral blood mononuclear cells in both humans and mice^{38,39}, although the variability is greater among humans³⁸. Most importantly, the supernatant of human iNKT cells activated by 7DW8-5 was found to be able to display anti-viral activity in vitro, and the activity was inhibited by anti-human IFN-γ antibody (FIGS. 20A-B). The parental glycolipid, α-GalCer, has been administered (6 IV doses of 0.12 mg/kg) to cancer patients without evidence of toxicity⁴⁰. In a vaccine adjuvant study in rhesus macaques, up to 100 μg of 7DW8-5 intramuscularly did not lead to any adverse reactions⁴¹, similar to the lack of toxicity noted in mice and hamsters in the current study.

Current COVID-19 vaccines have played a tremendous role in mitigating the impact of the pandemic, by decreasing symptomatic infections, hospitalizations, and deaths. The excessive fear that was once rampant has largely dissipated due to the development of these vaccines as well as effective treatments. However, as SARS-COV-2 has continued to evolve antigenically, breakthrough infections have become common⁴², resulting, at a minimum, in major disruptions to our daily lives. Additional prevention modalities are therefore urgently needed. In some embodiments, 7DW8-5 is another tool in our fight against COVID-19. Furthermore, this glycolipid could also be useful for addressing future outbreaks or pandemics caused by an emergent respiratory virus long before vaccines or drugs are developed.

### Materials and Methods

### Data Reporting

Power analysis based on guidelines by Institute for Laboratory Animal Research were used to predetermine sample size to estimate minimum number of animals required to detect significant effect of 7DW8-5 glycolipid, if one is detected. The experiments were not randomized, and the investigators were not blinded to allocation during experiments and outcome assessment.

### Animals

Female BALB/c mice with 10-15 weeks of age and female C57BL/6 mice with 14-15 weeks of age, as well as female BALB/c mice lacking CD1dl and CD1d2 genes (strain: C.129S2-Cd1tm1Gru/J) and female C57BL/6 mice lacking IFN-γ (strain: B6.129S7-Ifngtm 1Ts/J) were purchased from The Jackson Laboratory (Bar Harbor, ME) and maintained under specific pathogen-free conditions in the animal facility at Columbia University Irving Medical Center. Heterozygous 8-9 weeks old female K18-hACE C57BL/6J mice (strain: 2B6.Cg-Tg (K18-ACE2) 2Prlmn/J) were obtained from The Jackson Laboratory and housed in a pathogen-free animal facility at Washington University School of Medicine. Male Syrian hamsters with 5-6 weeks of aged were purchased from Charles River Laboratories (Wilmington, MA) and housed in an enhance biosafety level 3 (BL3) facility at Washington University in St. Louis.

### Glycolipids

Glycolipid 7DW8-5 with chemical formula [(2S,3S,4R)-1-O-(α-D-galactopyranosyl)-N-(11-(4-fluorophenyl) undecanoyl)-2-amino-1,3,4-octadecanetriol)] was synthesized as previously described⁵. Glycolipid α-galactosyl ceramide (α-GalCer) with chemical formula [N-[(3S,4R)-3,4-dihydroxy-1-[(2S,3R,4S,5R,6R)-3,4,5-trihydroxy-6-(hydroxymethyl)oxan-2-yl]oxyoctadecan-2-yl]hexacosanamide] and control lipids with chemical formula 18:1 caproylamine PE [1,2-dioleoyl-sn-glycero-3-phosphoethanolamine-N-(hexanoylamine)] and 18:1 biotinyl PE [1,2-dioleoyl-sn-glycero-3-phosphoethanolamine-N-(biotinyl) sodium salt] were purchased from Avanti Polar Lipids.

### Cells and Viruses

Vero-E6 (ATCC Cat #1586) and Vero-TMPRSS2-ACE2 cells (gift from Emory University) were cultured at 37 oC in Dulbecco's Modified Eagle medium (DMEM) supplemented with 10% fetal bovine serum (FBS), 10 mM HEPES pH 7.3, and 100 U/ml of penicillin-streptomycin. The SARS-COV-2 MA10, Delta variant (B.1.617.2) and Omicron variant (B.1.1529) viruses were obtained from BEI (cat #NR-55329, NR-55691 and NR-56475). Omicron stock was propagated in Vero-TMPRSS2 cells as described³ while other SARS-COV-2 isolates were propagated in Vero E6 cells for this study as described⁴³. All work with infectious SARS-COV-2 was performed in approved BSL3 and A-BSL3 facilities at Columbia University Irving Medical Center or at Washington University using appropriate positive pressure air respirators and protective equipment. Madin-Darby Canine Kidney (MDCK) cells (Cat #CCL-34) and HEp-2 cells (Cat #CCL-23) were purchased from the ATCC. Influenza A virus (H1N1) PR/8/34 strain (Cat #VR-95) and human respiratory syncytial virus (RSV) A2 isolate (Cat #VR-26PQ), purchased from ATCC were propagated and titrated in MDCK and Hep-2 cells, respectively under BSL2 condition.

### Nasal Turbinate and Lung Homogenates

Nasal turbinate was harvested from mice as described⁴⁴. Briefly, the skin was dissected and completely removed from the skull and nose, and the skull sectioned in the coronal plane. Then the remainder of the anterior skull was removed. After removing the posterior aspect of the skull base, the scissor was inserted into the posterior nasal cavity. The suture line was then incised bilaterally revealing the nasal septum. The scissor separated the two maxillas (laterally) and the ethmoid bone (medially) along the distinct suture line. Finally, the upper palate and anterior nasal tip were removed to complete the dissection. For the nasal turbinate from hamster, nasal turbinate was harvested by first removing the skin along the side of the nose and cheeks. The jaw was then cut exposing the hamster's palate. A sagittal incision through the palate was made exposing the nasal turbinate which was removed via blunt forceps. The nasal turbinate was homogenized in 1 mL DMEM supplemented with 2% FBS, L-glutamine and 1% HEPES. Homogenate was centrifuged at 1,000× g for 5 minutes. Whole lung tissue was weighed after separation from bronchial tubes and other non-lung tissues, placed in BioMasher II tube (Diagnocine LLC) with 250 μL of DMEM media supplemented with 2% heat-inactivated FBS and manually homogenized by rotating the grinder back and forth until all the tissue was thoroughly homogenized. Afterwards, the tissue homogenates in the tubes were centrifuged at 1000× g at 4-10° C. for 10 min. For both nasal turbinate and lung homogenates, the supernatant was collected and stored at-80° C. for viral titer and viral load detection.

### Tissue Viral Load Assays

Lung and nasal turbinate homogenates of 7DW8-5- or saline-treated and challenged mice and hamsters collected in the ABSL3 facility was used to set up the TCID50 titration assay. Serial 2-fold dilution of the samples were added to a 96-well plate seeded with 2×10⁴ Vero-E6 cells at 37° C. under 5% CO⁴³. Three days later, the wells were visually scored for cytopathic effect (CPE, as observed by light microscopy) of the cells at each dilution, to determine the end point TCID. For the PFU assay, Vero-TMPRSS2-ACE2 cells were seeded at a density of 1.25×10⁵ cells/well in flat-bottom 24-well tissue culture plates. The following day, media was replaced with 200 microliters of 10-fold serial dilutions of sample, diluted in DMEM+2% FBS. One hour later, 1 mL of methylcellulose overlay was added. Plates were incubated for 72 h, then fixed with 4% paraformaldehyde (final concentration) in PBS for 1 hour. Plates were stained with 0.05% (w/v) crystal violet in 20% methanol and washed twice with distilled, deionized water. Plaques were counted, and titers were calculated according to a previously described method⁴⁵.

For RSV titration, lung homogenates of saline or 7DW8-5 treated and challenged mice were serially diluted by 4-fold and overlaid on confluent layer of Hep-2 cells³². A virus control standard of the A2 isolate used to infect the mice was also serially diluted and run in parallel to compare the resulting CPE. Infected and control cells were incubated at 37° C. under 5% CO2 for 5 days. Viral CPE was observed under microscope for each dilution. Using the endpoint dilution method, the TCID50 was calculated per treatment group.

For influenza PR8 virus titration, lung homogenates of saline- or 7DW8-5-treated mice were harvested on day −5 post challenge with 200 PFU of PR8/A/34 virus per mouse. Homogenates were serially diluted by 3-fold and overlaid on a confluent layer of Martin Delaney Canine kidney (MDCK) cells. The PR8/A/34 isolate also serially diluted and used as positive control. Cells were incubated at 33° C./5% CO₂ and supernatants were collected at 72 h to determine viral neuraminidase activity from each well using a fluorescence assay^{46,47} with the NA-Fluor Influenza Neuraminidase Assay kit (Applied Biosystems). Production of fluorogenic product 4-methylumbelliferone was read in a Spectramax i3X microplate reader using excitation wavelength of 360nm and emission wavelength of 450nm. Negative controls using uninfected cell supernatants serve as blank for the assay. Endpoint for each sample was calculated as 3-fold or higher increase of the neuraminidase activity compared to the controls. TCID/g of lung was calculated for each sample and plotted using GraphPad Prism v9.3.

### Treatment With Anti-IFN-γ Antibody

For IFN-γ neutralization, anti-mouse IFN-γ antibody (BioXCell; clone XMG1.2) or a rat IgG1 isotype control (BioXCell; clone HRPN) was administered to mice by intraperitoneal injection at Day-1 (0.5 mg) and Day 0 (0.5 mg) relative to MA10 inoculation.

### Harvesting Bronchoalveolar Lavage

Bronchoalveolar lavage (BAL) was harvested as described⁴⁸. Briefly, after euthanizing the mouse, the animal was placed on its back on a surgical plate. After making an incision in the neck skin near the trachea using a scalpel, the trachea surrounded by sternohyoid muscle was exposed. After placing a cotton thread under the trachea using pincers, the middle of the exposed trachea between two cartilage rings was carefully punctured with a 26 G needle. Then, the catheter of about 0.5 cm was inserted into the trachea and stabilized by tying the trachea around the catheter using the cotton thread placed. A 1 mL syringe loaded with 1 mL of sterile balanced salt solution with 100 μM EDTA was connected to the catheter and the salt/EDTA solution was gently injected into the catheter. The solution was then gently aspirated while massaging the thorax of the mouse. The syringe was removed from the needle and the recovered lavage fluid was transferred into a 15 mL tube placed on ice.

### Cytokine and Chemokine Measurements

Sera, as well as supernatants were collected by centrifugation of nasal turbinates (NT), bronchioalveolar lavage (BAL), and lung homogenates (LH) of mice treated with 7DW8-5 one day before. Sera and supernatants were also collected from mice treated with saline one day before. The sera and supernatants were analyzed for cytokines and chemokines by Eve Technologies Corporation (Calgary, AB, Canada) using their Mouse Cytokine/Chemokine 31-Plex Array (MD31).

### Harvesting Lung Mononuclear Cells

Lung mononuclear cells (MNCs) were harvested as described⁴⁹. As shown in FIG. 17A, lungs were first perfused with PBS containing collagenase IV and DNasel. Single cell suspensions were prepared by mechanical dissociation of lung tissue through a 70-μm nylon mesh, followed by being layered on Ficoll-Paque (Sigma) and centrifuged at room temperature for 20 min at 900 g. After collecting lung MNC from the gradient interphase, the cells were incubated with anti-mouse Fc receptor antibody (BioLegend, Cat #101320) to block nonspecific antibody binding. Each sample was then stained with phycoerythrin-labeled anti-mouse CD45 antibody (BioLegend, Cat #103106) for 30 min. Just before sorting cells, DAPI (×10,000 dilution) was added. Viable cells were sorted by FACS Aria II by gating DAPI negative CD45 positive cells, followed by analyzing using FlowJo v10.8.1 software, as shown in FIG. 17B.

Single Cell 5′ transcript and TCRαβ Enriched Library Preparation on the 10× Chromium and Sequencing

The scRNA-Seq and scTCR-Seq libraries were prepared using the 10× Chromium single-cell 5′ reagent kits v2 (Dual Index), per manufacturer's instructions. In brief, after cell sorting, single-cell suspensions were loaded into the Chromium controller to make nanoliter-scale droplets with uniquely barcoded 5′ gel beads called GEMs (gel bead-in emulsions). After GEM-RT, GEMs were cleaned up by Dyna beads MyOne Silane beads (Thermo Fisher Scientific, 37002D). According to the manufacturer's instructions, the cDNA was amplified and size-selected by SPRI-beads (Beckman Coulter, B23317). Finally, the 5′ transcript and TCR-seq libraries were pooled and sequenced with the Illumina NovaSeq 6000 system.

Data Processing of scRNA-seq and TCR-seq

FASTQ files were processed by Cell Ranger v.6.1.2 (10× Genomics) software using the GRCm38 (GENCODE vM23/Ensembl 98) genome as a reference. The data analyzed in R using the Seurat v.4.1.0 package were merged and integrated using an anchor-based single-cell data integration method in Seurat⁵⁰. Cells with mitochondrial RNA content greater than 5% were excluded. Variable feature genes were set at 2000 genes. Once the data sets were integrated, the data were input into a principal component analysis (PCA) based on variable genes. The same principal components were used to generate the Uniform Manifold Approximation and Projections (UMAPs). Clusters were identified using shared nearest neighbor-based (SNN-based) clustering. For the clustering analysis, the function RunUMAP, FindClusters, and FindNeighbors in Seurat were used. Single cell TCR sequencing was aligned and quantified using the cellranger-vdj software (v.6.1.2). For the data analysis, the output file filtered_contig_annotations.csv was used.

### REFERENCES

1. Baden, L. R. et al. Efficacy and Safety of the mRNA-1273 SARS-COV-2 Vaccine. N Engl J Med 384, 403-416, doi: 10.1056/NEJMoa2035389 (2021).
2. Polack, F. P. et al. Safety and Efficacy of the BNT162b2 mRNA Covid-19 Vaccine. N Engl J Med 383, 2603-2615, doi: 10.1056/NEJMoa2034577 (2020).
3. Iketani, S. et al. Antibody evasion properties of SARS-COV-2 Omicron sublineages. Nature 604, 553-556, doi: 10.1038/s41586-022-04594-4 (2022).
4. Liu, L. et al. Striking antibody evasion manifested by the Omicron variant of SARS-COV-2. Nature 602, 676-681, doi: 10.1038/s41586-021-04388-0 (2022).
5. Li, X. et al. Design of a potent CD1d-binding NKT cell ligand as a vaccine adjuvant. Proc Natl Acad Sci USA 107, 13010-13015, doi: 10.1073/pnas. 1006662107 (2010).
6. Hammond, J. et al. Oral Nirmatrelvir for High-Risk, Nonhospitalized Adults with Covid-19. New England Journal of Medicine 386, 1397-1408, doi: 10.1056/NEJMoa2118542 (2022).
7. Jayk Bernal, A. et al. Molnupiravir for Oral Treatment of Covid-19 in Nonhospitalized Patients. New England Journal of Medicine 386, 509-520, doi: 10.1056/NEJMoa2116044 (2021).
8. Taylor, P. C. et al. Neutralizing monoclonal antibodies for treatment of COVID-19. Nature Reviews Immunology 21, 382-393, doi: 10.1038/s41577-021-00542-x (2021).
9. Edwards, A. M., Baric, R. S., Saphire, E. O. & Ulmer, J. B. Stopping pandemics before they start: Lessons learned from SARS-COV-2. Science 375, 1133-1139, doi: doi: 10.1126/science.abn1900 269 (2022).
10. Kronenberg, M. Toward an understanding of NKT cell biology: progress and paradoxes. Annu Rev Immunol 23, 877-900, doi: 10.1146/annurev.immunol.23.021704.115742 (2005).
11. Bendelac, A., Rivera, M. N., Park, S. H. & Roark, J. H. Mouse CD1-specific NK1 T cells: development, specificity, and function. Annu Rev Immunol 15, 535-562, doi: 10.1146/annurev.immunol.15.1.535 274 (1997).
12. Godfrey, D. I., Uldrich, A. P., Mccluskey, J., Rossjohn, J. & Moody, D. B. The burgeoning family of unconventional T cells. Nat Immunol 16, 1114-1123, doi: 10.1038/ni.3298 (2015).
13. Renukaradhya, G. J. et al. Type I NKT cells protect (and type II NKT cells suppress) the host's innate antitumor immune response to a B-cell lymphoma. Blood 111, 5637-5645, doi: 10.1182/blood-279 2007-05-092866 (2008).
14. Schmieg, J., Yang, G., Franck, R. W. & Tsuji, M. Superior protection against malaria and melanoma metastases by a C-glycoside analogue of the natural killer T cell ligand alpha-Galactosylceramide. J Exp Med 198, 1631-1641, doi: 10.1084/jem.20031192 (2003).
15. Wu, L. & Van Kaer, L. Natural killer T cells and autoimmune disease. Curr Mol Med 9, 4-14, doi: 10.2174/156652409787314534 (2009).
16. Gonzalez-Aseguinolaza, G. et al. alpha-galactosylceramide-activated Valpha 14 natural killer T cells mediate protection against murine malaria. Proc Natl Acad Sci USA 97, 8461-8466, doi: 10.1073/pnas.97.15.8461 (2000).
17. Brigl, M. et al. Innate and cytokine-driven signals, rather than microbial antigens, dominate in natural killer T cell activation during microbial infection. J Exp Med 208, 1163-1177, 290 doi: 10.1084/jem.20102555 (2011).
18. Tupin, E. et al. NKT cells prevent chronic joint inflammation after infection with Borrelia burgdorferi. Proc Natl Acad Sci U S A 105, 19863-19868, doi: 10.1073/pnas.0810519105 (2008).
19. Brossay, L. et al. CD1d-mediated recognition of an alpha-galactosylceramide by natural killer T 294 cells is highly conserved through mammalian evolution. J Exp Med 188, 1521-1528, doi: 10.1084/jem.188.8.1521 (1998).
20. Kawano, T. et al. CD1d-restricted and TCR-mediated activation of valpha14 NKT cells by glycosylceramides. Science 278, 1626-1629, doi: 10.1126/science.278.5343.1626 (1997).
21. Kakimi, K., Guidotti, L. G., Koezuka, Y. & Chisari, F. V. Natural killer T cell activation inhibits hepatitis B virus replication in vivo. J Exp Med 192, 921-930, doi: 10.1084/jem. 192.7.921 (2000).
22. Ishikawa, H. et al. IFN-gamma production downstream of NKT cell activation in mice infected with influenza virus enhances the cytolytic activities of both NK cells and viral antigen-specific CD8+ T 302 cells. Virology 407, 325-332, doi: 10.1016/j.virol.2010.08.030 (2010).
23. Littwitz-Salomon, E., Schimmer, S. & Dittmer, U. Natural killer T cells contribute to the control of acute retroviral infection. Retrovirology 14, 5, doi: 10.1186/s12977-017-0327-8 (2017).
24. Fujii, S., Shimizu, K., Smith, C., Bonifaz, L. & Steinman, R. M. Activation of natural killer T cells by alpha-galactosylceramide rapidly induces the full maturation of dendritic cells in vivo and thereby acts as an adjuvant for combined CD4 and CD8 T cell immunity to a coadministered protein. J Exp Med 198, 267-279, doi: 10.1084/jem.20030324 (2003).
25. Hermans, I. F. et al. NKT Cells Enhance CD4<sup>+</sup>and CD8<sup>+</sup>T Cell Responses to Soluble Antigen In Vivo through Direct Interaction with Dendritic Cells. The Journal of Immunology 171, 5140-5147, doi: 10.4049/jimmunol. 171.10.5140 (2003).
26. Paget, C. et al. Potential role of invariant NKT cells in the control of pulmonary inflammation and CD8+ T cell response during acute influenza A virus H3N2 pneumonia. J Immunol 186, 5590-5602, doi: 10.4049/jimmunol. 1002348 (2011).
27. Guillonneau, C. et al. Combined NKT cell activation and influenza virus vaccination boosts memory CTL generation and protective immunity. Proc Natl Acad Sci U S A 106, 3330-3335, doi: 10.1073/pnas.0813309106 (2009).
28. Johnson, T. R., Hong, S., Van Kaer, L., Koczuka, Y. & Graham, B. S. NK T cells contribute to expansion of CD8(+) T cells and amplification of antiviral immune responses to respiratory syncytial virus. J Virol 76, 4294-4303, doi: 10.1128/jvi.76.9.4294-4303.2002 (2002).
29. Leist, S. R. et al. A Mouse-Adapted SARS-COV-2 Induces Acute Lung Injury and Mortality in Standard Laboratory Mice. Cell 183, 1070-1085 e1012, doi: 10.1016/j.cell.2020.09.050 (2020).
30. Halfmann, P. J. et al. SARS-COV-2 Omicron virus causes attenuated disease in mice and hamsters. Nature 603, 687-692, doi: 10.1038/s41586-022-04441-6 (2022).
31. Ying, B. et al. Protective activity of mRNA vaccines against ancestral and variant SARS-CoV-2 strains. Sci Transl Med 14, eabm3302, doi: 10.1126/scitranslmed.abm3302 (2022).
32. Stokes, K. L. et al. Differential pathogenesis of respiratory syncytial virus clinical isolates in BALB/c mice. J Virol 85, 5782-5793, doi: 10.1128/JVI.01693-10 (2011).
33. Toapanta, F. R. & Ross, T. M. Impaired immune responses in the lungs of aged mice following influenza infection. Respir Res 10, 112, doi: 10.1186/1465-9921-10-112 (2009).
34. Shtrichman, R. & Samuel, C. E. The role of gamma interferon in antimicrobial immunity. Curr Opin Microbiol 4, 251-259, doi: 10.1016/s1369-5274 (00) 00199-5 (2001).
35. Chong, Z. et al. Nasally delivered interferon-lambda protects mice against infection by SARS-COV-2 variants including Omicron. Cell Rep 39, 110799, doi: 10.1016/j.celrep.2022.110799 (2022).
36. Shiratsuchi, T., Schneck, J., Kawamura, A. & Tsuji, M. Human CD1 dimeric proteins as indispensable tools for research on CD1-binding lipids and CD1-restricted T cells. J Immunol Methods 345, 49-59, doi: 10.1016/j.jim.2009.04.002 (2009).
37. Zhou, D. et al. Robust SARS-COV-2 infection in nasal turbinates after treatment with systemic neutralizing antibodies. Cell Host Microbe 29, 551-563 e555, doi: 10.1016/j.chom.2021.02.019 340 (2021).
38. Montoya, C. J. et al. Characterization of human invariant natural killer T subsets in health and disease using a novel invariant natural killer T cell-clonotypic monoclonal antibody, 6B11. Immunology 122, 1-14, doi: 10.1111/j. 1365-2567.2007.02647.x (2007).
39. Lee, Y. J. et al. Tissue-Specific Distribution of iNKT Cells Impacts Their Cytokine Response. Immunity 43, 566-578, doi: 10.1016/j.immuni.2015.06.025 (2015).
40. Giaccone, G. et al. A phase I study of the natural killer T-cell ligand alpha-galactosylceramide (KRN7000) in patients with solid tumors. Clin Cancer Res 8, 3702-3709 (2002).
41. Padte, N. N. et al. A Glycolipid Adjuvant, 7DW8-5, Enhances CD8+ T Cell Responses Induced by an Adenovirus-Vectored Malaria Vaccine in Non-Human Primates. PLOS ONE 8, e78407, doi: 10.1371/journal.pone.0078407 (2013).
42. Dance, A. Omicron's lasting mysteries: four questions scientists are racing to answer. Nature 603, 352 22-24, doi: 10.1038/d41586-022-00428-5 (2022).
43. Wang, P. et al. Antibody resistance of SARS-COV-2 variants B.1.351 and B.1.1.7. Nature 593, 130-135, doi: 10.1038/s41586-021-03398-2 (2021).
44. Antunes, M. B. et al. Murine nasal septa for respiratory epithelial air-liquid interface cultures. BioTechniques 43, 195-204, doi: 10.2144/000112531 (2007).
45. Case, J. B., Bailey, A. L., Kim, A. S., Chen, R. E. & Diamond, M. S. Growth, detection, quantification, and inactivation of SARS-COV-2. Virology 548, 39-48, doi: 10.1016/j.virol.2020.05.015 (2020).
46. Wetherall, N. T. et al. Evaluation of neuraminidase enzyme assays using different substrates to measure susceptibility of influenza virus clinical isolates to neuraminidase inhibitors: report of the neuraminidase inhibitor susceptibility network. J Clin Microbiol 41, 742-750, doi: 10.1128/JCM.41.2.742-750.2003 (2003).
47. Eichelberger, M. C., Hassantoufighi, A., Wu, M. & Li, M. Neuraminidase activity provides a practical read-out for a high throughput influenza antiviral screening assay. Virol J 5, 109, doi: 10.1186/1743-193 422X-5-109 (2008).
48. Au-Van Hoecke, L., Au-Job, E. R., Au-Saelens, X. & Au-Roose, K. Bronchoalveolar Lavage of Murine Lungs to Analyze Inflammatory Cell Infiltration. JoVE, e55398, doi: doi: 10.3791/55398 (2017).
49. Chang, S. H. et al. T helper 17 cells play a critical pathogenic role in lung cancer. Proc Natl Acad Sci USA 111, 5664-5669, doi: 10.1073/pnas. 1319051111 (2014). 50. Stuart, T. & Satija, R. Integrative single-cell analysis. Nat Rev Genet 20, 257-272, doi: 10.1038/s41576-199 019-0093-7 (2019).

### Example 4—Histology/Immunohistochemistry

### Results

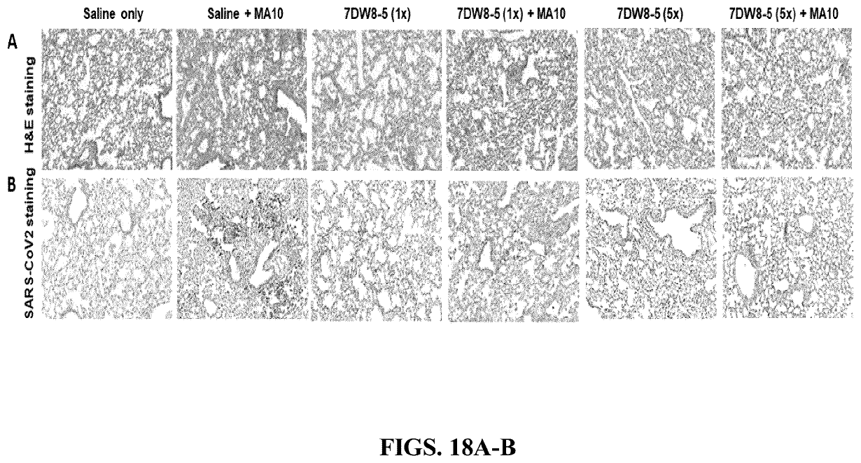

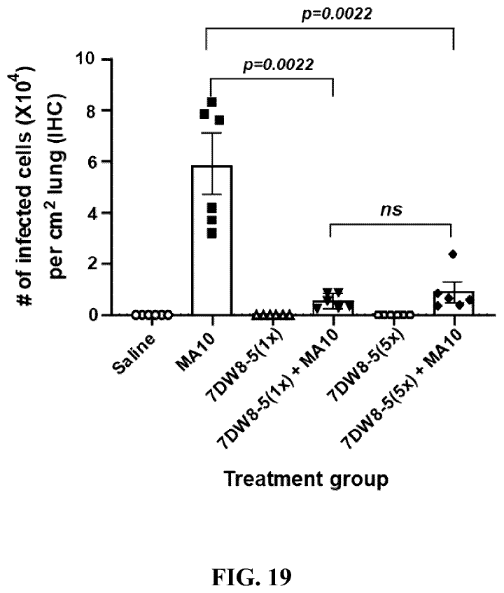

Lung histopathologic analyses with hematoxylin and eosin (H&E) staining before and after MA10 challenge revealed early multifocal damage with an accumulation of inflammatory cells, sloughed epithelial cells, and plasma proteins in the airway lumens (FIG. 18A). Treatment with 7DW8-5 prevented such lung tissue damage. Immunohistochemistry (IHC) staining for SARS-COV-2 nucleocapsid revealed an intense staining at 3-day post infection in the lung tissue of a saline-treated and MA10-challenged mouse, whereas there was little staining in the lung tissues of mice treated with a single or repeated dosing of 7DW8-5 (FIG. 18B). In fact, when the number of infected spots were counted, both single and multiple dosing of 7DW8-5 significantly reduced the number of spots (FIG. 19), consistent with the observed reduction in lung viral titers shown by the TCID50 assay.

### Method

Histological and immunohistochemical assays were conducted as described^{1,2}. Immediately after euthanasia, the left lung lobe was harvested and fixed by submersion in 10% phosphate buffered formalin for overnight, followed by submersion in 70% ethanol for up to one week, which was enough to disinfect the viruses. Fixed tissue was manually embedded in paraffin, and sections were prepared at 4 μm thickness. Tissue sections were stained with hematoxylin and eosin (Richard Allan Scientific, San Diego, CA). For immunohistochemistry, antigen retrieval was performed with antigen unmasking solution (pH 6.0 citrate buffer, H-3300, Vector Laboratories, Newark, CA), followed by peroxide incubation. The tissue sections were first incubated with primary antibody, SARS-COV-2 nucleocapsid protein rabbit mAb (26369, Cell Signaling, Danvers, MA) for overnight, and then with secondary antibody, HRP-conjugated anti-rabbit IgG antibody (MP7401, Vector Laboratories). The signal was then amplified using a DAB staining kit (NC9567138, Fisher Scientific, Waltham, MA) and it was counterstained with hematoxylin. Finally, tissues were tiled scanned using an Aperio AT2 slide scanner (Leica Biosystems, Deer Park, IL), and images were captured with an Aperio ImageScope (Leica Biosystems, Deer Park, IL).

### References for Example 4

1. Leist, S. R., et al. A Mouse-Adapted SARS-COV-2 Induces Acute Lung Injury and Mortality in Standard Laboratory Mice. Cell 183, 1070-1085 e1012 (2020).
2. Mori, M., et al. Generation of functional lungs via conditional blastocyst complementation using pluripotent stem cells. Nat Med 25, 1691-1698 (2019).

### Example 5—Human Relevance

### Results

In some embodiments, the subject matter described herein relates to the treatment of respiratory viral infections in humans. Supernatants collected from human iNKT cell lines stimulated with CD1d-transfected Hela cells in the presence of 7DW8-5, but not 18:1 PE, were able to significantly inhibit ic-SARS-COV-2-mNeon infection in Huh7 cells in vitro, and that this inhibition was completely abolished when neutralizing anti-human IFN-γ Ab was added to the culture (FIGS. 20A-B). All these results indicate a significant role that IFN-γ plays for mediating anti-SARS-COV-2 activity by 7DW8-5 both in mice and humans.

### Methods

Human iNKT cell lines and HeLa cells transfected with the human CD1d gene, HeLa-hCD1d, were established, as we previously reported¹. Twenty thousand human iNKT cell lines from 3 different donors were co-cultured with 2×10⁴ HeLa-hCD1d in a well of 96-well round-bottom plate in the presence of 1 μg/mL of 7DW8-5 glycolipid, 18:1 PE or saline (controls). After 24 h incubation, the cultured supernatants were collected and estimated for presence of IFN-γ using ELISA. Supernatants collected from stimulated human iNKT cell lines were serially diluted (2-fold) and incubated on a monolayer of Huh7 cells overnight at 37° C./5% CO₂ for 20 h. A serial 5-fold dilution of recombinant human IFN-γ protein was used as positive control (Gibco cat #PHC4031). In addition, supernatants were combined and mixed with anti-human IFN-γ antibody (1 μg/mL) (BioXcell Cat #BE0235) to check for specific and non-specific effects. Following this incubation, the cells were infected with ic-SARS-COV-2-mNeon isolate and incubated further for 24 h under similar conditions. After 24 h, cells were examined under fluorescent microscope to enumerate GFP+ cells and percent inhibition was calculated as the difference in fluorescence in the test wells compared to wells receiving no treatment (virus only) and plotted using GraphPad Prism v10.

### REFERENCE

1. Li, X. et al. Design of a potent CD1d-binding NKT cell ligand as a vaccine adjuvant. Proc Natl Acad Sci USA 107, 13010-13015, doi: 10.1073/pnas. 1006662107 (2010).”

1. A method of treating or preventing a respiratory infection in a subject in need thereof, the method comprising administering to the subject a pharmaceutical composition comprising a therapeutically effective amount of a glycolipid.

2. The method of claim 1, wherein the glycolipid is 7DW8-5, wherein 7DW8-5 is administered in a dosage of about 0.5 μg/kg. 1 μg/kg. 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

3. (canceled)

4. The method of claim 1, wherein the glycolipid is α-GalCer or an α-GalCer analogue.

5. (canceled)

6. The method of claim 1, wherein the method comprises activation of iNKT cells, NK cells, CD8+ cells, CD4+ cells, B cells, or combinations thereof.

7-10. (canceled)

11. The method of claim 1, wherein the method comprises increased secretion of one or more cytokines compared to the secretion of the same one or more cytokines in a non-treated subject, wherein a cytokine of the one or more cytokines is interferon-γ.

12. (canceled)

13. The method of claim 1, wherein the administering route is intranasal, intravenous, oral, intramuscular, or intradermal.

14-19. (canceled)

20. The method of claim 1, wherein the respiratory infection is caused by:
a) a SARS-COV-2 virus;
b) an influenza virus; or
c) a respiratory syncytial virus (RSV).

21-76. (canceled)

77. A method of activating natural killer T (NKT) cells in a subject in need thereof, the method comprising administering to the subject a pharmaceutical composition comprising a therapeutically effective amount of a glycolipid.

78. The method of claim 77, wherein the glycolipid is 7DW8-5, wherein 7DW8-5 is administered in a dosage of about 0.5 μg/kg, 1 μg/kg, 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg. about 95 μg/kg, about 100 μg/kg, about 110 μg/kg, about 120 μg/kg, about 130 μg/kg, about 140 μg/kg, or about 150 μg/kg.

79. (canceled)

80. The method of claim 77, wherein the glycolipid is α-GalCer or an α-GalCer analogue.

81. (canceled)

82. The method of claim 77, wherein the natural killer T cells comprise iNKT cells.

83-84. (canceled)

85. The method of claim 77, wherein the administering route is intranasal, intravenous, oral, intramuscular, subcutaneous, or intradermal.

86-91. (canceled)

92. The method of claim 77, wherein the activation is mediated via one or more cytokines, wherein a cytokine of the one or more cytokines is interferon-γ.

93. (canceled)

94. The method of claim 77, wherein the activation of T cells is localized to lungs or nasal turbinates.

95. (canceled)

96. The method of claim 77, wherein the method comprises treating or preventing a respiratory infection in the subject, wherein the respiratory infection is a SAR-Cov-2 infection, a respiratory syncytial virus (RSV) infection, or an influenza infection.

97-101. (canceled)

102. A method of stimulating an innate immune response in a subject in need thereof, the method comprising administering to the subject a pharmaceutical composition comprising a therapeutically effective amount of a glycolipid.

103. The method of claim 102, wherein the glycolipid is 7DW8-5, wherein 7DW8-5 is administered in a dosage of about 0.5 μg/kg, 1 μg/kg. 2 μg/kg, 3 μg/kg, 5 μg/kg, about 7 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, or about 150 μg/kg.

104. (canceled)

105. The method of claim 102 wherein the glycolipid is α-GalCer or an α-GalCer analogue.

106. (canceled)

107. The method of claim 102, wherein the immune response comprises inducing iNKT cells, NK cells, CD8+ T cells, CD4+ T cells, B cells, or combinations thereof.

108-111. (canceled)

112. The method of claim 102, wherein the administrating route is intranasal, intravenous, oral, intramuscular, subcutaneous, or intradermal.

113-118. (canceled)

119. The method of claim 102, wherein the inducing is mediated via one or more cytokines, wherein a cytokine of the one or more cytokines is interferon-γ.

120. (canceled)

121. The method of claim 102, wherein the stimulating is localized to lungs or nasal turbinates.

122. (canceled)

123. The method of claim 102, wherein the method comprises treating or preventing a respiratory infection in the subject, wherein the respiratory infection is a SAR-COV-2 infection, a respiratory syncytial virus (RSV) infection, or an influenza infection.

124-153. (canceled)

154. The method of claim 77, wherein the activating is localized to lungs or nasal turbinates.

155. The method of claim 77, wherein the method comprises treating or preventing a respiratory infection in the subject, wherein the respiratory infection is a SAR-COV-2 infection, a respiratory syncytial virus (RSV) infection, or an influenza infection.
